(19) United States
(12) Patent Application Publication
(10) Pub. No.: US 2005/0148500 A1
(43) Pub. Date: Jul. 7, 2005

(54) Dopaminergic stimulatory factor

(76) Inventors: Alfred Heller, Chicago, IL (US); Lisa Won, Burr Ridge, IL (US); Martin Gross, Chicago, IL (US)

Correspondence Address: FULBRIGHT & JAWORSKI L.L.P., 600 CONGRESS AVE., SUITE 2400, AUSTIN, TX, 78701, US

(21) Appl. No.: 10/496,913

(22) PCT Filed: Nov. 26, 2002
(86) PCT No.: PCT/US02/37946

Related U.S. Application Data
(60) Provisional application No. 60/333,561, filed on Nov. 27, 2001.
(60) Provisional application No. 60/411,806, filed on Sep. 18, 2002.

(51) Int. Cl. A61K 48/00; A61K 38/17

(52) U.S. Cl. 514/12; 424/93.21

(57) ABSTRACT

The present invention provides a novel proteinaceous composition that increases the levels of dopamine (DA) in neurons. Therefore, provided are methods for treating conditions caused by the deficiency of dopamine and/or by the loss of DA neurons or injury to DA neurons. An example includes Parkinson's disease. Also provided are methods for isolating and partially purifying the proteins and further characterizing the same.

The present application claims priority to co-pending U.S. Patent Application Ser. No. 60/333,561, filed Nov. 27, 2001, and co-pending U.S. Patent Application Ser. No. 60/411,806, filed Sep. 18, 2002. The entire text of each of the above-referenced disclosures is specifically incorporated by reference herein without disclaimer.

The government owns rights in the present invention pursuant to grant number MH 28942 from the National Institutes of Mental Health and grant number 17-01-1-0819 from the DAMD.

### BACKGROUND OF THE INVENTION

1. Field of the Invention

The present invention relates generally to the fields of neurobiology and neurodegenerative diseases involving dopaminergic neurons. More particularly, it concerns the identification of proteinaceous factors that increase the dopamine content of cells capable of expressing dopamine. The factors of the present invention have therapeutic potential for diseases relating to dopamine deficiency such as Parkinson's disease.

2. Description of Related Art

Parkinson's disease (PD) is a common affliction affecting some 1.0% of individuals over the age of 55. In the United States alone, PD afflicts over a million individuals. PD is a progressive debilitating disorder with a variety of motor dysfunctions including rigidity, bradykinesia and tremor. Those afflicted with PD suffer considerable motor and psycological disability and eventual death. The prevalence, morbidity and mortality associated with Parkinson's disease (PD) has stimulated an intense investigation of a variety of strategies for the treatment of this disorder.

PD is associated with a marked loss in dopamine (DA) secondary to degeneration of the nigrostriatal DA projection. Interestingly, the signs and symptoms of PD in humans and in experimental models of the disease (Ungerstedt, 1971; Schwarting and Huston, 1996; Hudson el al., 1993) are only evident after an approximately 80% or greater neuronal loss of the DA projection (Hornykiewicz and Kish, 1987). For this reason, individuals with fairly marked loss of DA neurons can still maintain quite satisfactory extrapyramidal motor function. Therefore, therapeutic interventions which can prevent or retard DA cellular loss such as increasing sprouting of DA axons or upregulating the DA phenotype of the remaining nigrostriatal neurons have the potential for reversing the motor deficits.

A variety of therapeutic approaches are available including L-dihydroxyphenylalanine (L-DOPA) therapy, cellular transplants, neurosurgical intervention and treatment with antioxidants. The L-DOPA therapy is largely ineffective long-term and has numerous side-effects. Another approach is to treat PD by the application of neurotrophic agents. Such substances are present in the nigrostriatal projection and support the normal survival and differentiation of mesencephalic DA neurons (for review see Beck (1994) and Hefti (1997) and references therein). About fourteen such “trophic agents” have been identified and a variety have been tested in animal models of the disease. Of these, glial derived neurotrophic factor (GDNF) has been reported to enhance the survival of human fetal DA neurons and implants of fetal cells (Mendez et al., 2000; Meyer et al., 1999). Clinical trials of this trophic agent in PD patients are, in fact, in progress (Alexi et al., 2000). However, since the GDNF-based treatment requires the use of human fetal cells this raises several ethical questions and concerns.

Given the possibility that trophic agents, which are proteins, may be of utility in treatment, there has been considerable interest in devising strategies for CNS delivery. These approaches include intracerebral injection as well as the encapsulation of cells which are engineered to continuously synthesize and secrete trophic agents and their implantation into the lateral ventricle (Apfel, 1997). Another approach is the intraparenchymal injection of a replication-defective adenoviral vector encoding GDNF which when injected near the substantia nigra has been found to protect DA neurons from progressive degeneration induced by striatal injection of 6-hydroxydopamine (Choi-Lundberg et al., 1997). Recent studies have shown that the conjugation of GDNF to the transferrin receptor (OX26) provides a means of “piggybacking” to permit access across the blood brain barrier. This strategy has been useful in affecting motor neuron survival in intraocular grafts (Albeck et al., 1997) and as a means of providing nerve growth factor to cholinergic striatal cells (Kordower et al., 1994).

However, what the art lacks is a trophic factor/agent that either increases the dopamine content of neurons and/or directly allows dopaminergic neurons to survive and produce dopamine.

### SUMMARY OF THE INVENTION

The present invention overcomes these and other defects in the art and provides proteinaceous trophic factors that markedly increase the levels of dopamine in primary neuronal cultures as well as in dopaminergic immortalized cells. Therefore, the present invention provides methods that can successfully treat and/or prevent conditions that result from a deficiency of dopamine and/or from the degeneration of dopaminergic neurons. Hence, using the methods of the present invention one can prevent and/or provide therapy for PD, as well as other neurological conditions involving dopamine deficiency or loss of dopaminergic neurons.

The present invention relates to compositions comprising at least one proteinaceous factor that is isolatable from X61 cells and has the ability to increase the dopamine content in neurons. In some cases, the proteinaceous factor is further defined as heat-labile and/or trypsin-sensitive and/or having a molecular weight of less than 100 Kda. In some cases, the neurons are dopaminergic neurons. These include, but are not limited to, primary dopaminergic neurons and immortalized dopaminergic neurons.

In some embodiments, the factor is partially purified or substantially purified. Further, the composition may be isolated from cells in culture, produced synthetically, produced by recombinant methods, or obtained by any other method known to those of skill in the art.

In other embodiments, the invention contemplates methods for the preparation of peptide mimetics that have all or some of the same characteristic activities of the native proteinaceous compositions of the inventions. Mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of an antibody and antigen. A peptide mimetic is expected to permit molecular interactions similar to the natural molecule. It is an aspect of the invention to engineer second generation molecules having many of the natural properties of native proteinaceous compounds according to the invention, but with altered and even improved characteristics, in conjunction with the principles outline above. Those of ordinary skill in the art will recognize that one can, in view of the teachings of this application, prepare and test candidate mimetics for activity and utility. For one of ordinary skill in the art, such a mimetic is easy to make or obtain. Such mimetic peptides would be very easy for a clinician to test. In some embodiments, the mimetic may be administered directly to a subject. In some cases, the mimetic may be constructed or modified to have improved stability, for example, modified to be non-hydrolyzable for oral administration.

The invention also contemplates the fusion of the proteinaceous compositions and/or mimetics to carriers that will assist in the composition in crossing the blood brain barrier. For example, ferritin can be used as a carrier. Those of ordinary skill will be able to prepare and test such fusions.

The inventions further relates to methods for providing therapy and/or preventing a condition caused by a deficiency of dopamine and/or a loss or injury of dopaminergic neurons comprising administering to a patient a composition comprising at least one proteinaceous factor, mimetic, or fusion according to the invention that has an ability to increase the dopamine content in neurons. In some preferred embodiments, the condition is Parkinson's disease or schizophrenia. Administration may be via any method known to those of skill in the art, including but not limited to intravenous, intra-portal, intra-arterial, intracerebral, or direct local injection or oral administration.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Effect of 48 h incubation with X61 protein on MN9D dopamine levels. The data are expressed as the mean±SEM of n=5 cultures per group. *P<0.001, two-tailed t-test, as compared to vehicle control (distilled water).

FIG. 2. Effect of 5 day incubation with X61 protein on dopamine content per MN9D cell. The data are expressed as the mean±SEM of n=5 cultures per group. *P<0.001, two-tailed t-test, as compared to vehicle control (distilled water).

FIG. 3. Effect of protein on primary mesencephalic dopaminergic neurons grown in reaggregate culture with nontarget tectal cells. Cultures were treated for 20 days with 0.8 mg/ml of protein obtained from N18TG2 or X61 cells, or with distilled water (vehicle control). The results are expressed as the mean±SEM of t=5 for N18TG2 and X61 treated cultures, and n=4 for control. *P<0.002, two-tailed t-test, as compared to control.

### DESCRIPTION OF ILLUSTRATIVE EMBODIMENTS

The prevalence, morbidity and mortality associated with Parkinson's disease has resulted in the development of a variety of strategies for the treatment of this disorder (Alexi et al., 2000). However, Parkinsonian symptoms in humans are only evident after an approximately 80% neuronal loss in this projection system (Hornykiewicz and Kish, 1987). Behavioral measures of dopamine loss in experimentally-induced rodent models of Parkinson's disease, such as drug-induced rotation, are only observed after a 90% or greater depletion of dopamine, whereas deficits in forelimb function are evident with reductions in transmitter of only 75% (Lindner et al., 1995). Thus, the onset of PD symptoms in humans or experimental animals is a result of a loss of a significant population of dopaminergic neurons. Hence, therapeutic interventions that can prevent or retard dopaminergic cellular loss are aimed at increasing sprouting of dopaminergic axons or upregulating the dopaminergic phenotype of remaining nigrostriatal neurons.

By somatic cell hybridization techniques the present inventors have previously produced immortalized monoclonal hybrid cells that are dopaminergic (Wainwright et al., 1995). This was achieved by the fusion of cells from the corpus striatum, the area of brain suffering a loss of innervation during PD with the N18TG2 neuroblastoma, (Wainer et al., 1992). Several immortalized cell lines were produced.

In the present invention, the inventors have discovered that one of the immortalized monoclonal lines, X61, produces a composition comprised of at least one or more protein(s) which markedly increases the dopamine content of a dopaminergic cells exemplified by a cell line derived from the mesencephalon (MN9D) and primary dopaminergic neurons in culture. The proteinaceous compositions of the invention produce a marked increase in the level of dopamine, for example, a six-fold increase in dopamine level is seen in of monolayer cultures of MN9D cells exposed to about 2.4 mg/ml of the X61-derived protein for about 48 h. The dopaminergic stimulatory activity is located in the cytosol of the X61 cells and has been observed in cell supernatant obtained upon the gentle disruption of the X61 cells. Therefore, the invention provides a proteinaceous composition or a trophic agent/factor that is capable of increasing the production of dopamine and hence, capable of increasing dopaminergic cell survival.

Therefore, the invention provides methods for treating and/or preventing neurodegenerative diseases involving dopamine deficiency and dopamine neuronal degeneration by providing to a subject the proteinaceous composition of the invention.

Characterizing the mechanisms of action of the proteinaceous composition on cellular dopamine levels and on dopamine synthesis at the morphological, neurochemical and genetic level will lead to new insights on the therapeutic aspects of the invention. Additionally, the invention provided methods to isolate, purify and determine the amino acid sequence(s) of the protein(s) comprised in the proteinaceous composition of the invention. As will be appreciated by one of skill in the art, the identification of the proteinaceous composition will also lead to identifying of the nucleic acid sequences that encode the composition. Hence, the invention also provides methods for providing gene therapy comprising administering to a subject a nucleic acid encoding the proteinaceous composition of the invention.

A. Neurodegenerative and Other Neurological Conditions Attributed to Dopamine Levels

The basal ganglia play a central role in the integration of information from the limbic system and neocortex (for reviews see Bannon and Roth, 1983; Graybiel and Ragsdale, 1983; Graybiel, 1990; Gerfen, 1992). The involvement of the basal ganglia in sensorimotor function is evidenced by the neurodegenerative diseases such as Parkinson's disease and Huntingtons's chorea (Hornykiewicz, 1973, 1979; Seeman et al., 1989). Dysfunction of mesocortical dopaminergic systems has also been implicated in neuropsychiatric disorders including schizophrenia (Seeman, 1987; Carlsson, 1988; Seeman et al., 1993).

A number of neuronal compartments within the corpus striatum have been defined on the basis of their morphology, afferent projections, or neurochemical phenotype (Graybiel, 1990; Gerfen, 1992; Stoof et al., 1992). The regulation of synthesis and release of neurotransmitters within each of these compartments is under complex control of several pathways including the nigrostriatal dopaminergic projection to the medium spiny neurons which comprise the majority of the neuronal population of the corpus striatum.

The effects of the neurotransmitter dopamine within the corpus striatum are mediated by at least two dopamine receptor subtypes, D₁ and D₂. Dopamine receptors have been classified as D₁-like (including D₁ and D₅) and D₂-like (including D₂, D₃, and D₄) receptor families based on their molecular sequence and pharmacological properties (for review see Kebabian and Calne, 1979; Andersen et al., 1990; Grandy and Civelli, 1992; Sibley and Monsma, 1992; Gingrich and Caron, 1993).

The mechanisms underlying DA neuronal survival and function are complex. DA neurons of the mesencephalon and their axonal processes comprise a number of projection systems to the telencephalon (Moore and Bloom, 1978). The DA neurons which degenerate in PD, represent, at most, 1% of the cells of the mesencephalon and this subpopulation is made up of DA cells differing in their topographic arrangement, neurochemistry and terminal targets. Thus, the population of neurons and glia of target areas affected in PD such as the corpus striatum and cortex are very heterogenous.

B. Immortalized Neuronal Cell Lines

As heterogeneous populations of neurons are present in one given area of the brain it is difficult to study the effects on a single sub-population of neurons. In order to circumvent these difficulties, cell lines expressing specific neurotransmitter phenotypes have been developed (Wainer et al., 1992). Such cell lines yield unlimited numbers of homogeneous cell populations for cellular and molecular investigations of the regulatory mechanisms controlling neuronal survival and differentiation, and the establishment and maintenance of appropriate cell-cell interactions. In addition, monoclonal immortalized cell lines expressing specific trophic factors provide an unlimited cell source for the isolation and purification of active agents.

The present inventors have previously used methods of somatic cell fusion to immortalize mature neurons expressing different neurotransmitter phenotypes in order to obtain monoclonal cell lines to characterize neurotrophic factors expressed by such cells (Wainer et al., 1992). Typically, it is difficult if not impossible to identify the neuronal origin of a trophic factor in a given region of the brain that is generally composed of heterogeneous cell populations.

The methods used by the inventors to produce immortalized monoclonal neurons involved cell fusion of brain cells from the corpus striatum (which is the area of brain suffering a loss of innervation during PD) with the neuroblastoma N18TG2 cells, by exposure to polyethylene glycol (Wainwright et al., 1995, incorporated herein by reference in its entirety). N18TG2 neuroblastoma cells are inherently deficient in the enzyme hypoxanthine phosphoribosyltransferase. Hence, when cells subject to fusion are grown in media containing hypoxanthine, aminopterin and thymidine only the fused cells survive (Wainer et al., 1992). This approach permitted the generation of monoclonal hybrid cells derived from neurons of the septohippocampal and nigrostriatal projections expressing specific transmitter phenotypes (Wainer et al., 1992; Choi et al., 1991; Hammond et al., 1986; Lee et al., 1990; Wainwright et al., 1995). This generated twenty-seven immortalized monoclonal corpus striatum hybrid cell lines, including the cell lines X61, X52, X57, X58, and X62 among others (Wainwright et al., 1995). Of particular relevance to the present invention was the generation of the X61 cell line derived from the nigrostriatal system.

In addition, another immortalized dopaminergic neuron, the MN9D, provides a useful test system for the identification of dopaminergic trophic factors. For example, the DA phenotype of MN9D cells can be modified by a number of manipulations. The DA levels of MN9D cells can be increased by forskolin treatment and markedly downregulated by contact with primary cells from areas of the brain which are nontargets for the DA neuron, i.e., areas such as optic tectum which do not receive a DA innervation. When MN9D cells are grown in three-dimensional reaggregate culture with primary cells of the optic tectum they suffer a marked loss of tyrosine hydroxylase and DA (Choi et al., 1992). The fact that the DA levels of such cells can be modulated makes them a useful test system for the detection of specific factors which can affect the DA phenotype and may be present in only limited quantities in primary cells.

Other features of the immortalized monoclonal corpus striatum hybrid cell lines include the expression of some or all of the following: an array of dopamine receptor mRNA, as well as a dopamine and adenosine 3′5′-monophosphate-regulated phosphoprotein, M, 32,000 (DARPP-32) mRNA, functional D₁ and D₂ dopamine receptors, choline acetyltransferase activity, acetylcholine.

a) Other Methods for Producing Immortalized Neuronal Cells

Several other methods have been used in the art to generate neuronal cell lines. For example, spontaneous transformation events or the introduction of transforming oncogenes have been used to generate immortalized cell lines which provide unlimited amounts of tissue (Cepko, 1989; Lendahl and Mckay, 1990). Spontaneous transformation has provided a number of widely studied cell lines including the PC12 cell line arising from a rat pheochromocytoma (Greene and Tischler, 1976). Spontaneous generation of a human neuronal cell line (Ronnett et at., 1990) has also been reported but such transformations are a rare event.

Retroviral-mediated oncogene transduction is another method used to immortalize neuroblasts at a high rate of efficiency. However, this approach has not successfully produced cells which express a specific neurotransmitter phenotype (Cepko, 1999; Lendahl and Mckay, 1990; Eves et al., 1992).

Neuroblastoma and retinoblastoma cell lines which express dopamine receptors are available (Balmforth et al., 1986, 1988; Monsma et al., 1989; Sidhu and Fishman, 1990; Ivins et al., 1991; Lovenberg et al., 1991) in addition to the COS-1 line which is derived from renal epithelium (Steffey et al., 1991). However, while such cell lines are homogenous and provide an unlimited supply of tissue for biochemical analyses, they are not derived from the brain and therefore, are limited utility in the study of neuronal cell-cell interactions in specific brain regions.

Somatic cell fusion is a technique that permits the immortalization of postmitotic neuronal cells from brain regions which express specific neurotransmitter phenotypes (Hammond et al., 1986; Lee et al., 1990; Choi et al., 1991; Crawford et al., 1992). Furthermore, cell lines produced in this manner may be used as models for the examination of the molecular mechanisms governing the differential expression of specific neurochemical phenotypes (Choi et al., 1992). Somatic cell fusion has been used for the generation of adrenergic cell lines from the peripheral nervous system (Greene et al., 1975) as well as those of central nervous system lineage including septal, hippocampal (Hammond et al., 1986, 1990; Lee et al., 1990), and ventral spinal cord (Cashman, 1991) cell lines. In addition, this technique has been used to establish dopaminergic cell lines derived from embryonic murine mesencephalic neurons (Choi et al., 1991) as well as a rat mesencephalic cell line (Crawford et al., 1992).

b) Somatic Cell Hybridization Methods

The methods used to produce the immortalized cell lines including X61 and MN9D are set forth in this section.

Materials. Embryonic mice used were of the C57BL/6J strain. Pregnant mice were obtained from controlled matings in a closed colony derived from Jackson Laboratory stock. Mice were housed with a constant light-dark cycle of 12 hr and fed a breeding diet of mouse chow containing 10% fat (Teklab).

All solutions used in tissue culture were filtered through 0.22 Amicon sterile filters (Nalgene) and stored in sterile bottles at 4° C. Dulbecco's modified Eagle medium (DMEM), fetal bovine serum (FBS), trypsin, and penicillin-streptomycin (5000 units penicillin; 5000 units streptomycin per ml) were obtained from Grand Island Biological Company. DNase was obtained from Worthington Biochemical. Media for cell lines in monolayer culture consisted of DMEM plus 10% (v/v) FBS and 1% (v/v) penicillin-streptomycin unless stated otherwise. Forskolin, R(+) SKF-38393, Quinpirole HCI, (R+) SCH-23390, and S(−) Eticlopride were obtained from Research Biochemicals International (Natick, Mass.).

Dissection and Somatic Cell Fusion. Embryonic day 18 C57BL/6J mouse embryos were employed as the source of primary corpus striatum cells. C57BL/6J cells express hypoxanthine phosphoribosyltransferase (HPRT) activity (Greene et al., 1975; Lee et al., 1990). The neuroblastoma fusion partner was the N18TG2 cell line, which is deficient in HPRT and therefore unable to utilize exogenous sources of hypoxanthine for purine synthesis (Greene et al., 1975). Following fusion, the cells were cultured in a medium containing hypoxanthine, aminopterin, and thymidine (HAT medium). As aminopterin blocks endogenous pathways required for purine and pyrimidine synthesis, the parent N18TG2 cells, which cannot utilize hypoxanthine, do not survive in this medium. Hybrid cells resulting from the fusion of N18TG2 and primary corpus striatum cells have the HPRT defect corrected by the inclusion of chromosomes from parental primary brain cells, thus enabling the hybrid cells to express HPRT and utilize exogenous sources of hypoxanthine. The neuroblastoma cell line was previously obtained through chemical mutagenesis of the N18 neuroblastoma, a subclone of the C1300 neuroblastoma isolated from an A/J mouse (Minna et al., 1975). The N18TG2 cell line expresses negligible levels of choline acetyltransferase (ChAT) activity (Greene et al., 1975; Hammond et al., 1986, 1990; Blusztajn et al., 1992).

Embryos were removed from C57BL/6J mice killed by cervical dislocation on the 18th day of gestation and staged according to the criteria of Gruneberg (1943). The embryonic brain dissection procedure has previously been described in detail (Hemmendinger et al., 1981). The dissociation of primary corpus striatum cells and their fusion with N18TG2 cells was carried out as previously described for the generation of septal cholinergic and mesencephalic dopaminergic cell lines (Hammond et al., 1986; Choi et al., 1991), using a modification of the procedure described by Fournier (1981). Briefly, dissociated CS cells were suspended in 2.5 ml of DMEM containing phytohemagglutinin-P (100 μg/ml) (Sigma, St. Louis). The cell suspension was then pipetted onto a 60 mm culture plate containing N18TG2 cells at approximately 70% confluence. After a 15 min incubation at 37° C., the medium was aspirated and the cells were exposed to 50% (v/v) polyethylene glycol (PEG 1000, Koch-Light) for 1 min at room temperature. These procedures are described in detail in Wainer (1992). The fusion products were incubated overnight in culture medium (DMEM supplemented with 10% fetal bovine serum) and replated in HAT medium to select for the expression of hybrid cells. Control fusions included N18TG2×N18TG2 and corpus striatum×corpus striatum cells. Neither of these fusions produced viable colonies. Therefore, any surviving colonies probably arose from hybrid cells derived from the fusion of corpus striatum cells with N18TG2 cells. One of the initial fusion products screened for specific ¹²⁵I-SCH 23982 binding to D₁ receptor binding sites was treated for 5d with a combination of (1 mM n-butyric acid and a specific D₁ dopamine receptor agonist (SKF 38393). This treatment was selected in an attempt to increase the expression of D₁ receptors in light of the earlier demonstration of the differentiating effects of n-butyric acid on cultured cells (Prasad and Sinha, 1976) and on somatic cell hybrid cell lines (Choi et al., 1991). In addition, previous studies (Monsma et al., 1990b) have shown that elevation of cyclic adenosine monophosphate (cAMP) using a membrane-permeable cyclic AMP analog leads to an increase in the expression of functional dopamine receptors in a retinoblastoma cell line. The use of a specific D₁ agonist was, therefore, intended to promote the expression of D₁ receptors by stimulation of adenylate cyclase consistent with the report of Monsma et al. (1990b) as well as that of Zhou et al. (1992) of the presence of a cAMP response element in the D₁ receptor gene While only 5% of the cells survived this treatment, it was possible to obtain a cell population (EIX) from the surviving colonies which does express D₁ and D₂ receptors. This cell population was then expanded and subcloned. Twenty-seven monoclonal cell lines were obtained and selected for further characterization.

Freezing of Cell Cultures. Cryostorage was performed as described previously for the freezing of mesencephalic hybrid cell lines (Choi et al., 1991). The freezing medium consisted of HEPES-buffered DMEM supplemented with 20% FBS and 10% (v/v) dimethyl sulfoxide (Fisher Scientific, Fair Lawn, N.J.).

b) Characterization of the Hybrid Cells

Membrane Preparation or Dopamine Receptor Characteriation. Cells were cultured on 100 mm Falcon plates. Media were aspirated and the monolayer cultures washed three times with harvesting buffer [50 mM Tris-HCl buffer (pH 7.7) at 5° C. with 5 mg NaEDTA and 120 mM NaCl]. After washing, the cells were harvested by mechanical disruption with a rubber policeman. The tissue was flash frozen in liquid nitrogen and stored at −70° C. Immediately prior to assay, tissue was thawed slowly on ice, and disrupted by homogenization (Heidolph homogenizer, Polyscience Corp, Niles, Ill.) with 10 up strokes at low speed in 20 volumes of harvesting buffer. The homogenate was centrifuged at 43,000×g for 20 min at 4° C. (Dupont, Sorvall RC28S with an SM24 head) and the resultant pellet suspended in 50 mM Tris HCl (pH 7.4 at 37° C.) with 120 mM NaCl.

Radioligand binding methods for D₁ and D₂ dopamine receptor binding sites were adapted from Sidhu and Kebabian (1985) as described previously (Farfel et al., 1992). D₁ binding sites were labeled with ³H-SCH 23390 (NEN Dupont, 80 Ci/mmol) or ¹²⁵I-SCH 23982 (NEN Dupont. 2200 Ci/mmol). D₂ binding sites were labeled with ³H-spiperone (NEN Dupont, 32.4 Ci/mmol). D₁ receptor binding sites were quantified using saturation studies (six or eight concentrations ranging from 0.02-0.9 nM). Aliquots of tissue (final concentration 100-300 μg protein/ml) were incubated with increasing concentrations of radioligand for 45 min at 37° C. in a final volume of 240 μl. Specific binding was defined by 100 μM fluphenazine. Ketanserin (10 μm) was included in all tubes to inhibit radioligand binding to 5-HT, serotonin receptor binding sites. For the D₂ assays, increasing concentrations of ³H-spiperone (six concentrations ranging from 0.04-0.7 nm) were incubated with aliquots of tissue (final concentration 75-250 μg protein/ml) for 45 min at 25° C. in Tris-HCl buffer (pH 7.5 at 25° C.) with 120 mM NaCl and 0.01% bovine serum albumin. Final assay volume was 300 μl. Specific binding was defined by 10 μM haloperidol. Ketanserin (10 μM) was included in all tubes to inhibit radioligand binding to 5-HT, serotonin receptor binding sites.

For all assays, incubation was terminated by filtration under reduced pressure over Whatman GF/B filters (pretreated with 0.3% poly-L-lysine) using a Brandel Cell Harvester modified for radioligand binding assays. Filters were rinsed three times with 5.0 ml ice-cold 50 mM Tris-HCl buffer (pH 7.7 at 25° C.). The filters were dried overnight and placed in disposable glass minivials (Research Products International). Three milliliters of a 95% Econofluor/5% Protosol solution (NEN, Waltham, Mass.) was added and the samples counted by liquid scintillation spectrophotometry (Beckman Model LS 5000TD) with an efficiency of 45%.

Screening of Fusion Products for Dopamine Receptor Binding Sites. Twenty-five cell populations produced by the initial fusion of N18TG2 and primary corpus striatum cells were screened for ¹²⁵I-SCH 23982 specific binding. Cells were cultured on 100 mm Falcon plates. Harvesting of the cells and membrane preparations for the binding assay were essentially as described by Monsma et al. (1989). The concentration of radioligand used was 0.6 nm.

Subcloning of the Fusion Products. One of the cell populations derived from the initial fusion of E18 corpus striatum with N18TG2 cells expressed high levels of ¹²⁵I-SCH 23982 binding sites. This cell population was treated with a combination of 1 mM n-butyric acid and a D1 agonist (SKF 39893, 10 μm) for 5 d. Less than 5% of the cells survived this treatment. One surviving population (E1X) was selected, expanded, and subcloned for further characterization. Subcloning of single cells from this colony was carried out using a modification of the single-cell plating technique (Puck et al., 1956) as described previously (Choi et al., 1991). Twenty-seven monoclonal cell lines were produced and characterized as described below.

HPLC Determination of Endogenous GABA Levels. Measurement of endogenous γ-aminobutyric acid (GABA) levels was performed as described previously (Kontur et al., 1987) using a modification of the method of Lasley et al. (1984). Briefly, cells were removed from the culture plates by mechanical disruption in 1 ml of 0.008 mg/ml GABA. The cell suspension was then sonicated and immediately stored at 4° C. An aliquot of the supernatant was derivatized with o-phthaldialdehyde and the indole derivative was subsequently separated and quantified by high-performance liquid chromatographic analysis with electrochemical detection (HPLC-ED). The chromatographic system consisted of a Milton Roy minipump (model 396), a 5 μM octadecyl 4.6×50 mm reverse-phase column (IBM), and an amperometric detector (BAS LC4) with a glassy carbon electrode (BASM-800). The mobile phase consisted of a sodium monobasic phosphate buffer (0.1 M) containing EDTA (0.134 M) and 35% (v/v) HPLC grade methanol at a pH of 5.6. The carbon electrode was maintained at a potential of +0.8 V versus the silver chloride reference electrode with a sensitivity of 20 nA/V.

Choline Acetyltransferase Activity and Endogenous Acetylcholine Content. Choline acetyltransferase (CHAT) activity was measured using a modification of the method of Fonnum (1975) (Hammond et al., 1986, 1990). ChAT specific activity was expressed as picomoles of acetylcholine formed per minute per milligram protein. Background signal was determined by assaying citrate-phosphate buffer instead of cell extract. Acetylcholine (ACh) levels were determined by HPLC-ED with an enzymatic reactor containing acetylcholinesterase and choline oxidase based on the method of Potter et al. (1983). The materials used were obtained from Bioanalytical Systems Ins. (West Lafayette, I).

Identification of Dopamine Receptor and DARPP-32 mRNA Expression by Polymerase Chain Reaction. Oligonucleotide primers were synthesized on an Applied Biosystems DNA synthesizer. Given the high degree of identity across species (for example, the mouse D₂ sequence shows 97% nucleic acid homology with the rat D₂ sequence; Mack et al., 1991), dopamine receptor-specific oligonucleotides used for this study were primarily derived from rat receptor sequences unless the mouse sequence was known. All of the chosen probes generated the predicted size fragment which was further verified using internal oligonucleotides as hybridization probes. For the D₁ receptor, sequences were derived from Monsma et al. (1990a) and included: o654, 5′-TCACTGCT CATCCTGTCCAC (SEQ ID NO. 1), identical to nucleotides 503-522; and o643, 5′ GAGCACATGATGTCAAAGGC (SEQ ID NO. 2), which is complementary to nucleotides 713-732. The D₁ receptor primers o197, o198 were as described by O'Malley et al. (1990). The D₁ receptor primers were derived from Sokoloff et al. (1990) and included: o580, 5′-TGGGCTATGGCATCTCTGAGTCAGCT (SEQ ID NO. 3), identical to nucleotides 76-101 and o198 noted above, which is also complementary to the rat D₁ nucleotides 400-423. The D₁ primers (o415, o416, o474) were as described (O'Malley et al., 1992). The D₁ (D₁B) primers were derived from Tiberi et al. (1991) and included o644, 5′-ACTGGGACCCGCGCAGGT (SEQ ID NO. 4), identical to nucleotides 99-119 and o643, the sequences of which are conserved between the D₁ and D₂ receptors (nucleotides 346-365 of the rat D₁ receptor, Tiberi et al., 1991). The DARPP-32 primers were 5′-CTGTGCCTATACGCCCCCATC (SEQ ID NO. 5) and 5′-GGGATGCTGAGGTTCCrCTCCAGGC TCAC (SEQ ID NO. 6).

RNA preparation and standardization were as described by Mack et al. (1991). PCR amplification and analysis were as described by O'Malley et al. (1990, 1992) for the D₁ and D₂ receptors, respectively. Temperatures for the D₁ PCR protocols were 93° C. for 90 s, 61° C. for 60 s, and 72° C. for 90 s. The D₁ and D₂ parameters were 93° C. for 60 s, 54° C. for 60 s, and 72° C. for 60 s. The DARPP-32 parameters were 94° C. for 60 s, 60° C. for 60 s, and 72° C. for 90 s. Oligonucleotides were end-labeled and then added to the PCR mixtures. Each template and primer set was optimized to ensure linearity of exposure. PCR products were separated on a 5% polyacrylamide gel which was subsequently dried and exposed to x-ray film.

Adenylate Cyclase Assay. Assays of receptor-mediated adenylate cyclase activity in corpus striatum hybrid cell membranes were modified from the original methodology (Childers, 1985) using ³H-ATP as a substrate. The H-cyclic AMP reaction product was quantitated by HPLC as described by Childers (1985). Previously frozen (−70° C.) tissue was homogenized in 50 volumes of ice-cold 50 mM Tris buffer (pH 7.7 at 4° C.) with 2 mM EGTA and 5 mM MgCl₂, with a glass-tefton homogenizer. Homogenates were centrifuged at 43,000×g for 20 min. The resultant pellets were resuspended in adenylate cyclase buffer (50 mM Tris-HCl with 5.0 mM MgCl₂, and 2 mM EGTA, pH 7.4 and kept on ice prior to use for adenylate cyclase assays. The membrane preparations were incubated with reaction mixture (160 μl) which contained 30 μM cyclic AMP, 10 μM ATP, 3 mM isobutylmethy L-xanthine, 5 mM creatine phosphate, 25 U/ml of creatine phosphokinase, 20 μM GTP, 0.04% bovine serum albumin, and 1 pCi ³H-ATP together with 25-50 μg membrane protein and various drug additions to a total volume of 240 μl. The reaction was initiated by the addition of the ³H-ATP, incubates at 30° C. for 10 min, and terminated by placing the tubes in a boiling water bath for 2 min. The tubes were cooled on ice for 5 min and 0.7 units of adenosine deaminase was added. After a 5 min incubation a 30° C., the tubes were transferred onto ice and the excess remaining ³H-ATP was removed by the addition of BaOH, and ZnSO₄ with 5 min between each addition. Blank levels were determined by boiling the tissue for 5 min before continuing with the assay. The tubes were centrifuged at 10,000×g for 15 min, and the supernatants transferred into a parallel set of tubes for automatic sample injection into the HPLC column. Each tube was run in triplicate and the means of the control and experimental tubes compared by a paired sample t-test.

HPLC apparatus (Beckman model 110B pump) was connected to a Rainin Microsorb 3 μM C-18 reverse-phase column together with a C-18 guard column. Mobile phase consisted of 0.8 M sodium acetate, pH 5.0, with 12% methanol at a flow rate of 1 ml/min. Samples were injected (220 μl volume) by a Spark Holland Marathon Autosampler which maintained sample temperature at 5-10° C. The cyclic AMP peak eluted 4.1 min after injection and was collected by a Varex model SF2120 fraction collector. Unlabeled cyclic AMP was detected at 254 nm through an 8 μl flow cell (Beckman Model 153 I: V Detector) with a sensitivity of 0.16 AU full scale. ³H-cyclic AMP, collected in three fractions of 0.25 ml was transferred to a glass scintillation vial to which 8 ml of CytoScint scintillation fluid (ICN Biomedicals) was then added. Radioactive decay was measured on a Beckman LS liquid scintillation counter at an efficiency of 45%. Under these conditions, more than 90% of the cyclic AMP was recovered as determined from studies using added ³H-cyclic AMP.

Aggregation Assays. The ability of the hybrid cells to aggregate in rotation mediated cell culture was tested as described by Choi et al. (1991), incorporated herein by reference, adapted from the procedure described by Moscona (1961), incorporated herein by reference, for examining cell-cell interactions. After 36 hr in culture, the aggregated cells were transferred to a depression slide and photographed.

Differentiation and Immunocytochemisty. The clonal hybrid cell lines X57, X58, and X62 and the parent neuroblastoma line N18TG2 were plated at a density of 5-20×10⁵ cells per 60 mm culture dish. The cells were maintained in culture for up to 5 d in the presence of 1 mM n-butyric acid, which causes some cultured cells to differentiate (Prasad and Sinha, 1976; Choi et al., 1991), or 10 μM forskolin, which maximally stimulates adenylate cyclase activity, or vehicle (0.1% dimethylsulfoxide). Cultures were examined for the expression of the neuronal marker neurofilament protein (NFP) and a glial marker, glial fibrillary acidic protein (GFAP). Immunostaining was performed according to the peroxidase-antiperoxidase method of Sternberger (1979) as described previously (Choi et al., 1991). The antibodies used were the monoclonal antibody 4.3F, which reacts with the carboxyl-terminal domains of the high-molecular-weight neurofilament subunits NFI50 and NF200 NFP, as well as 2.2B_{10.6} directed against GFAP (Trojanowski et al., 1983).

Protein Determination. Proteins were determined using the commercially available Pierce assay (Pierce, Rockford, Ill.) with bovine serum albumin as a standard.

Data Analysis. Saturation isotherms were fitted by a single-site model. Radioligand affinity (K_{d}) and the density of binding sites (Bₘₐₓ) were obtained using weighted Eadie-Hofstee plots as described by Zivin and Waud (1982). Computer-assisted analysis of ³H-SCH23390 and ³H-spiperone binding isotherms indicated a single, saturable site of interaction. Specific binding ranged from 50 to 80%, varying as a function of the concentration of radioligand and density of binding sites.

C. Neurotrophic Factors and the Proteinaceous Composition of the Invention

A number of the dopaminergic neurotrophic agents (Alexi et al., 2000), including GDNF and BDNF, are localized to the striatum and are, therefore, likely to be cellular products of either the neuronal or glial elements of this subdivision of brain. The production of immortalized monoclonal hybrid cells of neuronal origin was carried out, in part, with the objective of identifying unique dopaminergic neurotrophic agents/factors which might be present in the striatum and are difficult to identify or isolate in a heterogeneous cellular population. The proteinaceous composition obtained from the immortalized monoclonal X61 cell line that is capable of increasing the neurotransmitter content of dopaminergic neurons is a unique moiety differing from at least a substantial number of the known neurotrophic agents.

The X61-derived proteinaceous composition increases the cellular dopamine content of hybrid, monoclonal dopaminergic neurons of the mesencephalon as well as the dopamine content of three-dimensional reaggregate cultures containing primary mesencephalic dopaminergic neurons. Therefore, the proteinaceous composition is a candidate therapeutic agent that can increase the dopamine content of the nigrostriatal neurons which remain following either experimental or disease induced neuronal injury.

The present inventors have ruled out the possibility of the proteinaceous composition of the invention being some of the well known neurotrophic factors. For example, although western blot analysis revealed that X61 cells have low amounts of glial derived neurotrophic factor (GDNF) and ciliary neurotrophic factor (CNTF) in the X61-derived supernatant, exposure of MN9D cells to GDNF (1 ng/ml for 5 days) resulted in a significant decrease in dopamine levels as compared to controls (P<0.001). In the case of CNTF, 24-h treatment with 100 ng/ml had no significant effect on MN9D dopamine levels. Western blot analysis also shows no evidence for the presence in X61 supernatant of other trophic agents including brain derived neurotrophic factor (BDNF), neurotrophin 3, neurotrophin 4, insulin growth factor-I, fibroblast growth factor-1, bone morphogenetic factor-2, artemin, persephin, or neurturin.

D. Characterization and Isolation of the Protcinaccous Composition

The present invention also provides a composition that has the ability to increase the levels of dopamine in dopaminergic neuronal cells.

It is contemplated that the proteinaceous composition may comprise at least one peptide, a polypeptide, a protein or a small molecule which is associated with or bound to at least one peptide, polypeptide, or protein. The proteinaceous composition may additionally comprise a lipid moiety, and/or a carbohydrate moiety and/or a nucleic acid component. As the proteinaceous composition is heat-labile, it is contemplated to comprise at least one heat-labile peptide, polypeptide, or protein that may additionally be associated with or bound to at least one small molecule which may be a lipid moiety, and/or a carbohydrate moiety and/or a nucleic acid moiety.

Thus, in certain embodiments, the present invention concerns novel compositions comprising at least one proteinaceous molecule. As used herein, a “proteinaceous molecule,” “proteinaceous composition,” “proteinaceous factor,” “proteinaceous trophic factor,” “X61 protein(s),” “protein(s) of the invention” or “proteinaceous trophic agent” generally refers, but is not limited to, a polypeptide that is full length or less than full length that is translated from a gene; a polypeptide of of from about 3 to about 100 amino acids. All the “proteinaceous” terms described above may be used interchangeably herein. In some embodiments, it is expected that the proteinacious compositions of the invention may be cleavage products of a polypeptide of a larger size, such as a prohomone.

In certain embodiments, the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 amino acids. Further, the proteinacious molecule may be a cleavage product of a polypeptide of about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500, about 2500, about 3000, about 4000, about 4014, about 5000, about 10,000 or greater amino molecule residues, and any range derivable therein.

As used herein, an “amino molecule” refers to any amino acid, amino acid derivitive or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody. It is contemplated that antibodies to specific tissues may bind the tissue(s) and foster tighter adhesion of the glue to the tissues after welding. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this invention may be autologous proteins or peptides, although the invention is clearly not limited to the use of such autologous proteins As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject.

To select other proteins, polypeptides, peptides and the like for use in the methods and compositions of the present invention, one would preferably select a proteinacous material that possesses one or more of the following characteristics: it forms a solution with a high percentage of proteinaceous material solubilized; it possesses a high viscosity (i.e. about 40 to about 100 poise); it has the correct molecular charge to bind the dye if it is a non-covalent mixture (i.e. anionic protein and cationic dye, or cationic protein and anionic dye); it has the correct amino-acids present to form covalent cross-links (i.e. one or more tyrosines, histidines, tryptophans and/or methionines); and/or it is biocompatible (i.e. from mammalian origin for mammals, preferably from human origin for humans, from canine origin for canines, etc.; it is autologous; it is non-allergenic, and/or it is non-immunogenic).

a. Purification of the Proteinaceous Composition

As the proteinaceous composition of the invention has therapeutic benefits for Parkinson's disease and other neurological conditions involving dopaminergic neuronal degeneration or decreases in dopamine by increasing the dopamine content and the survival of dopaminergic neurons, it will be desirable to purify the proteinaceous composition. Methods for purifying proteinaceous are well known in the art. In some embodiments, it is contemplated that the proteinaceous composition may further be identified by purification methods such as those described below:

b. Purification of the Protein Component(s) of the Composition

It is contemplated that the proteinaceous composition of the invention may be isolated and identified. Protein purification and identification techniques are well known to those of skill in the art. These techniques involve, at one level, the crude fractionation of the cellular milieu to polypeptide and non-polypeptide fractions. Having separated the polypeptide from other proteins, the polypeptide(s) of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, isoelectric focusing, etc. A particularly efficient method of purifying peptides is fast protein liquid chromatography (FPLC) or even HPLC.

Certain aspects of the present invention concern the purification, and in particular embodiments, the substantial purification, of the protein or polypeptide or peptide comprising the proteinaceous composition of the invention. The term “purified protein or polypeptide or peptide” as used herein, is intended to refer to a composition, isolatable from other components, wherein the protein or polypeptide or polypeptide or peptide is purified to any degree relative to its naturally-obtainable state. A purified protein or polypeptide or peptide therefore also refers to a protein or polypeptide or peptide, free from the environment in which it may naturally occur.

Generally, “purified” refers to a composition comprising a protein or polypeptide or peptide that has been subjected to fractionation to remove various other components, and which composition substantially retains its expressed biological activity. Where the term “substantially purified” is used, this designation refers to a composition in which the protein or peptide forms the major component of the composition, such as constituting about 50%, about 60%, about 70%, about 80%, about 90%, about 95% or more of the proteins in the composition. The term “isolated”, when used to describe the composition disclosed herein, means protein that has been identified and separated and/or recovered from a component of its natural environment. Contaminant components of its natural environment are materials that would interfere with preventive or therapeutic uses for the protein, and may include other proteinaceous or non-proteinaceous solutes. In some embodiments, the protein may be purified (1) to a degree sufficient to obtain at least 15 residues of N-terminal or internal amino acid sequence, or (2) to homogeneity by SDS-PAGE under non-reducing or reducing conditions using Coomassie blue or, preferably, silver stain. “Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “fold purification number.” The actual units used to represent the amount of activity will, of course, be dependent upon the particular assay technique chosen to follow the purification and whether or not the protein or peptide exhibits a detectable activity. In the instant invention the proteinaceous composition comprising at least one heat-labile peptide, polypeptide, protein or a small molecule associated with at least one heat-labile peptide, polypeptide, or protein, having a molecular weight of less than 100 KDa can be detected by its activity that increases the levels of dopamine in primary cultures of dopamine neurons as well as in cultures of MN9D cells.

Various techniques suitable for use in protein purification are known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide comprising the proteinaceous composition always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) and FPLC are characterized by a very rapid separation with extraordinary resolution of peaks. This is achieved by the use of very fine particles and high pressure to maintain an adequate flow rate. Separation can be accomplished in a matter of minutes or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

It is contemplated that the proteinaceous compositions of the invention may be made by any technique known to those of skill in the art, including the expression of proteins, polypeptides or peptides through standard molecular biological techniques, the isolation of the proteinaceous compound from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques know to those of ordinary skill in the art. Alternatively, various commercial preparations of proteins, polypeptides and peptides are known to those of skill in the art.

E. Animal Models

Animal moels of diseases can be used to test the proteinaceous compositions of the invention in an in vivo system. One example is an aminal model of Parkinson's disease (PD). In general, the experimental models of PD are based on mechanical or drug-induced lesions of the nigrostriatal projection with resultant loss of components of the mesencephalic DA neuronal population (for an extensive review of this subject see Alexi et al. 2000, incorporated herein by reference in its entirety). Loss of such neurons with resultant terminal degeneration in the striatum, and concomitant reductions in the activity of striatal tyrosine hydroxylase and DOPA decarboxylase, and DA following transection of the nigrostriatal projection via the medial forebrain bundle (UTB) has been demonstrated by the inventors laboratory (Moore et al., 1971) and subsequently utilized to study the electrophysiological consequences of denervation of the corpus striatum (Levine et al., 1983; Levine et al., 1977; Levine et al., 1980). Such lesion induced degenerative models produced by mechanical or neurotoxic agents have been used for a wide variety of neurochemical, morphological and functional studies of the DA projection (Alexi et al., 2000).

Unilateral disruption of the nigrostriatal projection has a number of functional consequences which can be used to assess the effects of a variety of pharmacological agents and trophic factors. Animals with unilateral lesions show marked rotational responses to dopaminergic drugs (Alexi et al., 2000). Administration of DA agonists such as apomorphine and L-DOPA result in rotation contralateral to the lesion while indirect agonists such as amphetamine produce rotation ipsilateral to the lesion side. The mechanism of such unilateral effects are believed to be secondary to the lack of DA and the development of postsynaptic supersensitivity of DA receptors on the lesioned side of the brain. While the rotational model has been widely used to assess extrapyramidal function in experimentally induced PD, it can only be elicited by drug administration. Recent studies have utilized a motor test involving nondrug induced forepaw adjusting steps which has a number of advantages as a model for PD. This method was first introduced by Schallert et al. (1992) and subsequently utilized by Olsson et al. (1995) to examine the effects of pharmacological treatments and fetal mesencephalic grafts and by Winkler et al. (1996) to assess the effects of intranigral GDNF. The advantage of the use of the “stepping test” is that it is fairly simple to conduct and is spontaneously elicited by unilateral destruction of the dopaminergic projection. The test consists basically of moving a rat sideways along a table with only the forepaws touching the surface. A normal rat will take “steps” to adjust for the movement, but a unilaterally lesioned animal will show a severe deficit in “stepping” on the side of the body contralateral to the lesion at a threshold of 80% loss in dopamine (Chang et al., 1999). A variety of parameters can be measured, but the simplest is to count the number of steps taken. This deficit can be reduced by drugs and fetal dopamine cell grafting (Olsson et al., 1995).

In the present invention, it is contemplated that a well known aminal model of PD, such as those described above, can be tested by administration of the compositions of the invention. The ability of the instant compositions to treat or reduce the symptoms of disease can be assayed by methods known in the art.

F. Pharmaceutical Formulations and Delivery

In some embodiments of the present invention, methods for treatment for conditions associated with reduced DA levels or loss or injury of DA neurons are provided. These methods comprise administering to a patient in need thereof an effective amount of a pharmaceutical formulation of the proteinaceous compositions (also called the X61 protein(s)) of the invention.

An effective amount of the pharmaceutical composition, generally, is defined as that amount sufficient to detectably and repeatedly ameliorate, reduce, inhibit, minimize or limit the extent of the disease or its symptoms, or the extent of the condition, such as symptoms of PD, schizophrenia, etc. More rigorous definitions may apply, including elimination, eradication or cure of disease, or elimination or eradication of the symptoms.

Some useful doses contemplated for the amelioration of a disease include, but are not limited to, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0, or 10.0 mg protein per administration and all ranges between any two of these concentrations. Of course, any other dosage or dosage range may be employed, which dosage or dosage range can be determined by one of ordinary skill in the art employing known techniques.

The routes of administration will vary, naturally, with the location and nature of the disease or the condition, and include, e.g., parenteral, intravenous, intra-portal, intra-arterial, intracerebral, or direct injection. Local, regional or systemic administration also may be appropriate.

Continuous administration also may be applied where appropriate. Delivery may be via syringe or catherization. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on disease type, location, disease progression, and health and age of the patient. Obviously, certain types of diseases and conditions will require more aggressive treatment, while at the same time, certain patients cannot tolerate more taxing protocols. The clinician will be best suited to make such decisions based on the known efficacy and toxicity (if any) of the therapeutic formulations.

Solutions comprising the proteinaceous compositions of the invention may be constituted in pharmacologically acceptable solvents and may be prepared in water suitably mixed with a surfactant, such as hydroxypropylcellulose. Dispersions may also be prepared in glycerol, liquid polyethylene glycols, and mixtures thereof and in oils. Liposomal compositions are also contemplated. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions ([U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fingi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intraportal, intracerebral administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vaccuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

G. Clinical Trials

This section is concerned with the development of human treatment protocols for providing therapy to PD and other conditions involving deficiency in DA levels and/or loss or injury of DA neurons in a human patient using the proteinaceous compositions of the invention as described herein.

The various elements of conducting a clinical trial, including patient treatment and monitoring, will be known to those of skill in the art in light of the present disclosure. The following information is being presented as a general guideline for use in establishing the proteinaceous compositions of the invention, either alone or in combination with other standard therapies used in the art for the treatment of the neurological conditions that are treatable by the methods of the invention.

Candidates for the phase 1 clinical trial will be patients on which all conventional therapies have failed. Approximately 100 patients will be treated initially. Their age will range from 16 to 90 (median 65) years. Patients will be treated, and samples obtained, without bias to sex, race, or ethnic group.

Optimally the patient will exhibit adequate bone marrow function (defined as peripheral absolute granulocyte count of >2,000/mm³ and platelet count of 100,000/mm³, adequate liver function (bilirubin 1.5 mg/dl) and adequate renal function (creatinine 1.5 mg/dl).

The treatments described above will be administered to the patients regionally or systemically on a tentative weekly basis. A typical treatment course may comprise about six doses delivered over a 7 to 21 day period. Upon election by the clinician the regimen may be continued with six doses every three weeks or on a less frequent (monthly, bimonthly, quarterly etc.) basis. Of course, these are only exemplary times for treatment, and the skilled practitioner will readily recognize that many other time-courses are possible.

The modes of administration may be local administration, including, intracerebral administration. The mode of administration may be systemic, including, intravenous, intra-arterial.

In some embodiments, the proteinaceous compositions of the invention will be administered at dosages in the range including, but not limited to, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0, or 10.0 mg protein per administration and all ranges between any two of these concentrations. Of course, any other dosage or dosage range may be employed, which dosage or dosage range can be determined by one of ordinary skill in the art employing known techniques. In other embodiments the proteinaceous compositions of the invention may be administered as liposomal formulations. Of course, the skilled artisan will understand that while these dosage ranges provide useful guidelines, appropriate adjustments in the dosage depending on the needs of an individual patient factoring in disease, gender, age and other general health conditions, will be made at the time of administration to a patient by a trained physician. The same is true for means of administration and routes of administration as well.

To monitor disease course and evaluate the amelioration of the disease it is contemplated that the patients should be examined for appropriate tests every month. To assess the effectiveness of the proteinaceous compositions of the invention, the physician will determine parameters to be monitored depending on the type of disease and will involve methods to monitor the state of signs and symptoms of PD including the level of bradykinesia, extent of loss of rigidity, loss of motor control, and resting and intention tremors (in PD patients) and the like. Tests that will be used to monitor the progress of the patients and the effectiveness of the treatments include: physical exam, X-ray, blood work, and other clinical laboratory methodologies. The doses given in the phase I study will be escalated as is done in standard phase 1 clinical phase trials, i.e. doses will be escalated until maximal tolerable ranges are reached.

Clinical responses may be defined by acceptable measure. For example, a complete response may be defined by complete disappearance of the neurological disease or condition, whereas a partial response may be defined by a 50% reduction of the disease or condition.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### The Proteinaceous Composition Increases Dopamine in Neurons

A proteinacoeus composition, also referred to as “the X61 protein(s)” “the factor”, “the proteinaceous factor”, “the neurotrophic factor” or “the trophic factor”, of the invention, was obtained from an immortalized monoclonal line of striatal origin, the X61 cells. The factor is capable of increasing the dopaminergic content of a mesencephalic cell line, MN9D as well as of cultures containing primary dopaminergic neurons. The dopaminergic stimulatory activity was observed in the cell supernatant obtained following gentle disruption of X61 cells, an immortalized monoclonal cell line derived by fusion of the N18TG2 neuroblastoma with striatal neurons (Wainwright et al., 1995). The factor was obtained from X61 cells by washing once with 10 ml of calcium and magnesium-free Tyrode's solution (CMF) and removing the cells from the plates with a cell scraper. The cells were collected from the plates with 20 ml of CMF, transferred to a 50 ml centrifuge tube, centrifuged at 1000×g for 3 min and the pellets stored at −80° C. The pellets were broken up in 1 ml of sterile distilled water with a 22 gauge needle on a syringe which was used to draw the pellet up into the syringe 1105 times followed by similar treatment with a 25 gauge needle. The material was then transferred to a 1 ml sterile microcentrifuge tube on ice and centrifuged at 9000×g for 30 min at 4° C. The supernatant was removed and transferred to a 15 ml centrifuge tube, which was vortexed and 100 μl removed for protein determination (Smith et al., 1985). The supernatant obtained was stored at −20° C.

For the assessment of the stimulatory activity of the proteinaceous composition on dopamine content, MN9D cells were grown for 24 h on 100 mm tissue culture plates and then X61 supernatant (1.2 or 2.4 mg protein/ml) or sterile distilled water (vehicle) was added and the cultures incubated for 48 h. The MN9D cells on each plate were then collected in 10 ml of CMF, transferred to 15 ml centrifuge tubes and centrifuged at 1000×g for 5 min. The supernatant was removed and 600 μl of 0.5 N perchloric acid was added to the cell pellet which was then sonicated on ice for 10-15 s. The cell extract was centrifuged at 4000×g for 30 min and the supernatant stored at −80° C. for subsequent high pressure liquid chromatography (HPLC) analysis of MN9D cell dopamine content. The pellet was stored at −20° C. for subsequent protein analysis.

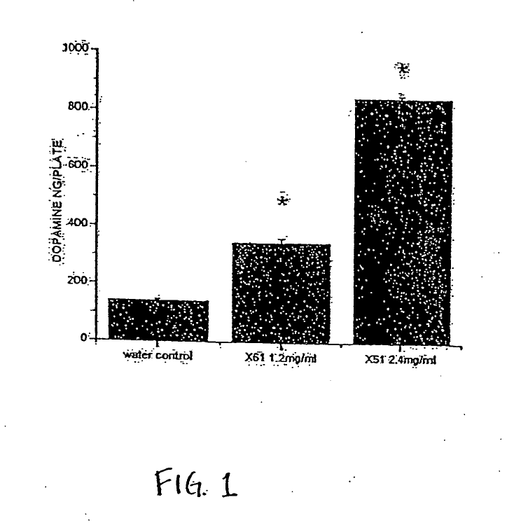

As can be seen in FIG. 1, the supernatant containing X61 protein, when applied for 48 h, produces a dose-dependent increase in the dopamine content of MN9D cultures. Significant increases in dopamine level are seen with 0.8 mg protein/ml of X61 supernatant even after 24 h of exposure (see below). Supernatant obtained from the N18TG2 cells, the cell partner used for somatic fusion with striatal neurons (Wainwright et al., 1995) was tested in two separate experiments one for 24 h and the other for 5 days of treatment and did not produce a significant increase in MN9D dopamine content (5 days of treatment: control: 281.5±17.0 ng/plate; N18TG2, 0.8 mg protein/ml: 341.3±45.2; and X61, 0.8 mg protein/ml: 543.6±82.7. Dopamine levels following treatment with X61 supernatant were significantly higher than control (P<0.02 two-tailed t-test). The active constituent of the X61 supernatant appears to be a protein since the activity is abolished by boiling at 100° C. for 30 min and can also be inactivated by exposure of the supernatant to trypsin. Preliminary sizing experiments using Amicon Centriprep concentrating filters indicate that the protein involved is less than 100 kDa. The protein does not appear to be secreted since medium conditioned by X61 cells has no effect on MN9D dopamine levels. The dopaminergic stimulatory activity seen with X61 protein appears to be specific to immortalized dopaminergic cells of central derivation since X61 protein had no effect on the dopamine content of peripherally-derived PC12 cells.

As can be seen in Table 2, X61 protein(s) when applied to MN9D cells for 48 hours produces a dose-dependent increase in MN9D cellular DA content. Significant increases in DA level are, in fact, seen with 0.8 mg protein/ml even after 24 hours of exposure. Protein obtained from cytosol of N18TG2 cells, the partner used for somatic fusion with striatal neurons (Wainwright et al., 1995) was tested in two separate experiments. One for 24 hours and the other for 5 days of treatment. In these experiments, the N18TG2 derived protein(s) did not produce any significant increase in the DA content of MN9D cells.

The DA stimulatory activity seen with X61 protein(s) appears to be specific to cells derived from central DA neurons since X61 protein(s) had no effect on the DA content of peripherally-derived PC12 cells.

TABLE 2
Effect of 48 hr Incubation with X61 Protein(s) on MN9D
Dopamine Levels

| | Treatment with | Treatment with |
|---|---|---|
| | 1.2 mg/ml | 2.4 mg/ml |
| Control | X61 protein | X61 protein(s) |
| 142.3 ± 5.5 | 343.3 ± 14.3* | 844.7 ± 16.0* |

Data expressed as ng dopamine mean±SEM per 100 mm culture dish. *p<0.001 two tailed t-test, n=5.

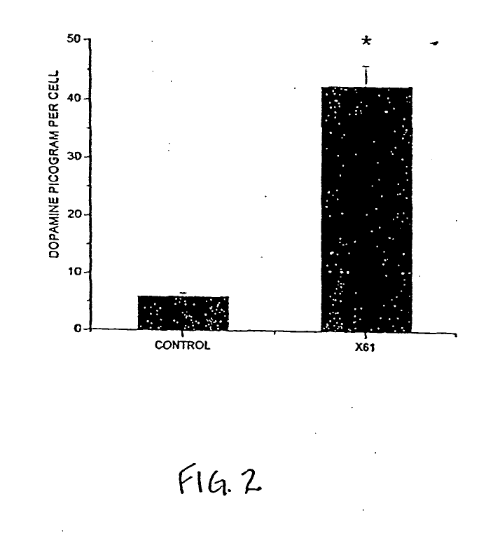

The finding of a six-fold increase in dopamine level of monolayer plates containing MN9D cells exposed to 2.4 mg/ml of X61 protein for 48 h is quite dramatic. A second experiment was then conducted to determine whether this effect was due to a change in the doubling time (24 h) of the MN9D cells with increased cell production or was the result of an increase in cellular dopamine. For that purpose, MN9D cells grown on 60 mm culture dishes were exposed to either distilled water or 1 mg/ml X61 protein for a period of 5 days. Cells were removed from the plates and cell numbers determined by counting in a hematocytometer. Dopamine content was determined by HPLC analysis. The MN9D cells can be differentiated by exposure to either sodium butyrate or dibutryl cyclic adenosine monophosphate for 5 days (Choi et al., 1991) resulting in a reduction in the rate of cell division and the production of extensive cellular processes. The MN9D cells exposed to X61 protein for 5 days did not differ in appearance from the controls and clearly had not undergone extensive morphological differentiation. However, fewer cells were present on the X61 protein treated plates as compared to control plates (control: 3.77±0.20 million cells; X61 treated: 1.85±0.14 million cells) suggesting a decrease in the rate of cell division. The effect on cellular dopamine was marked, with the X61 protein treated MN9D cells containing seven times more dopamine than the controls (FIG. 2). The result clearly demonstrates that X61 protein has a potent effect on the dopamine content of individual MN9D hybrid cells.

### Example 2

### Mechanism(s) of Increasing DA Content

To define the primary cellular mechanism by which X61 protein(s) increase the DA content of MN9D cells and coaggregates containing primary DA neurons the inventors contemplate the following experiments.

In coaggregates prepared from mesencephalon with tectal cells (which are nontargets for the DA neurons), a condition in which substantial numbers of the DA neurons fail to make axons and die off, the X61 protein(s) produce a three-fold increase in DA content of the coaggregate cultures. A similar result will be sought when X61 protein(s) is applied to aggregates composed of mesencephalon and striatum (the primary target for the nigrostriatal DA projection) a culture preparation in which there is an extensive formation of DA axons and quantitative survival of these cells. The fact that protein(s) obtained from striatal derived hybrid cells increases DA levels of mesencephalic-striatal aggregate cultures, in which the majority of DA neurons survive, would indicate that the effect of this protein(s) was to increase DA levels of individual DA neurons.

Three-dimensional reaggregate cultures containing primary DA neurons can be treated with X61 protein(s) and cell numbers and the DA content can be quantitated as a means of determining whether the effect is to increase DA content of individual cells or alternatively to increase axonal proliferation or actual cell survival.

Neurochemical Basis. To define the neurochemical basis of the increases induced by X61 protein(s) in DA content of individual MN9D cells one can proceed with examining a variety of neurochemical functions that lead to DA production including synthesis, degradation, storage of DA in the cells as well as uptake of released transmitter. Each of these neurochemical functions can be examined in MN9D cells in the presence and absence of X61 and N18TG2 protein(s).

For example, one can examine the effect of X61 protein(s) on cellular levels of tyrosine hydroxylase and DOPA decarboxylase, the enzymes required for DA synthesis in MN9D cells. Since cellular levels of DA can obviously be altered by mechanisms other than effects on biosynthesis, one can also examine the ability of X61 protein(s) to alter MN9D cell uptake and storage of DA. The results of these studies can be used to further investigations on the neurochemical status of primary DA neurons in aggregate culture and in the intact animal treated with X61 protein(s).

For the purposes of these studies, MN9D cells can be grown for 24 hours on 100 mm tissue culture plates and then soluble protein (1 mg/ml) from X61 cells, N18TG2 cells or sterile distilled water (vehicle) can be added and the cultures incubated for 48 hours. After this period, the MN9D cells may be analyzed for cellular DA content, as described above, as well as for the activity of enzymes involved in the biosynthesis and degradation of DA. A series of sensitive assays previously employed for the examination of the regional development of monoamines in the rat brain can be used (Porcher and Heller, 1972). The activity of tyrosine hydroxylase, the rate-limiting step in catecholamine biosynthesis, and DOPA decarboxylase can be assayed by methods described in Porcher and Heller (1972). Catechol-O-methyl transferase can be measured by a modification of the method of Zurcher and Da Prada (1982). Monoamine oxidase can be assayed using a modification of the method of Lewinshohn et al. (1978). Protein levels can be measured as described above.

Such studies provide discrete experimental data which can be used to pinpoint the specific neurochemical steps affected by X61 protein(s). Neurochemical changes following X61 protein treatment of MN9D cells can be extended to primary DA cells in aggregates and finally in an intact animal model.

Examination of Functional Changes in Gene Expression which may be Responsible for the Elevation of the DA Phenotype by the X61 Protein(s). One can also examine possible functional changes in gene expression which may be responsible for the elevation of the DA phenotype of the MN9D cell by X61 protein(s).

The inventors have already demonstrated that the DA phenotype of the MN9D cells can be downregulated by exposure of these cells to primary nontarget neurons of the optic tectum. Thus, the phenotype of these neurons are under some type of regulatory control. Agents which either increase or decrease DA levels in the MN9D cells may affect known protein transcription factors involved in the regulation of cellular DA phenotype. To this end, changes in early gene expression as well as the levels of a number of transcriptional factors involved in regulation of the DA phenotype can be assessed.

Assessment of the effect on gene expression can be examined by in situ hybridization in intact animals that are treated with the factor of the invention to see if similar gene regulation is observed in midbrain DA neurons. Additionally, one can identify the downstream signaling mechanism for this protein(s) and thereby identify other potential targets for therapeutic agents for conditions caused by DA deficiency.

While a generic screening for increased expression of c-fos mRNA can indicate a genetic response to the action of X61 protein(s) other molecular changes that are induced downstream from c-fos can also be determined. For example, one can determine whether a 24 hr exposure to X61 protein affects the mRNA and/or protein levels of tyrosine hydroxylase (TH), the enzyme which exerts rate-limiting control over DA synthesis. In addition, one can investigate whether exposure to X61 protein can affect the mRNA levels of c-fos (at times of 30, 60 and 90 min) and mRNA and protein levels of 3 factors (Nurr1, V-1 and Ptx3; at 24 hr and for extended times) which have been associated with the control of the DA phenotype.

Nurr1 is a member of the nuclear receptor family of transcription factors expressed in developing and mature DA neurons. Using transgenic Nurr1 knock-out mice, Saucedo-Cardenas et al. 1998 demonstrated that Nurr1 is involved in the later stages of development of DA neurons. In the absence of Nurr1, neuroepithelial cells that normally give rise to DA neurons are found to have a normal ventral localization and neuronal phenotype, but fail to develop a DA phenotype. This suggests that Nurr1 is involved in the commitment of mesencephalic precursor cells to the full DA phenotype. With further development, these precursor cells degenerate in the absence of Nurr1 suggesting that Nurr1 is also essential for survival.

A recent study (Yamakuni et al., 1998) has identified a novel protein containing cdc10/SW16 motifs which regulates mRNA levels of tyrosine hydroxylase, aromatic I-amino acid dearboxylase, dopamine β-hydroxylase, and phenylethanolamine N-methyltransferase. This protein, called V-1, is intensely co-expressed with TH protein in catecholamine-producing tissues in situ. Overexpression of V-1 in PC12 cells leads to an increase in DA levels of between 10-100 fold. V-1 is localized to the cytoplasm rather than the nucleus and it is predicted that the cdc/SW16 motif may serve to foster protein-protein interactions between V-1 and some unknown partner protein involved in transcriptional regulation. A homeodomain gene Ptx3 has also been identified whose expression is highly restricted to mesencephalic DA neurons (Smidt et al., 1997). Beginning at E11.5 in the developing mouse midbrain, the expression of Ptx3 correlates with the appearance of mesencephalic DA neurons. The number of neurons expressing Ptx3 is reduced in patients with Parkinson's disease and Ptx3-expressing neurons are also absent from 6-hydroxyDA-lesioned rats. Ptx3 appears to be a unique transcription factor for the mesencephalic DA neurons and may be involved in determining this neuronal lineage.

Specifically, one can examine levels of Nurr1, a member of the nuclear family of transcription factors involved in the later stages of development of the DA neuron, V-1, a novel protein which regulates mRNA levels of all of the enzymes involved in catecholamine biosynthesis, and, Ptx3, a homeodomain gene whose expression is restricted to mesencephalic DA neurons and may be reduced in Parkinson's disease. Ptx3 may also be involved in determining neuronal lineage. In addition, one can take advantage of recent advances in techniques for the analysis of differential gene expression to perform a more general analysis of other transcriptional changes possibly involved in the response of the MN9D cells and primary DA cells to exposure to X61 protein(s).

While studies on Nurr1, V-1 and Ptx3 can provide information on whether X61 protein regulates mRNA and protein levels of factors known to be involved in determination of the DA phenotype, they can not provide information on the levels of other regulators of DA function that may also be altered by treatment with X61 protein. The present inventors contemplate investigating what other genes might be regulated downstream from the actions of X61 protein and the elevation of immediate-early genes. Recent advances in techniques for the analysis of differential gene expression can be used to perform a more general analysis of the transcriptional changes involved in the response to X61 protein. There are a number of differential display methodologies which are roughly based on the same general steps, although some subtraction based approaches add steps specific to that technique. In general, high quality, undegraded mRNA is isolated from the two tissues to be compared. cDNA is produced by the reverse transcription of the mRNA normally using one-base anchored oligo-dT primers. Rounds of PCR using the anchored oligo-DT primers, or other primers, are used to amplify the cDNAs, followed by display of the cDNAs as bands on a gel. Differentially displayed cDNAs are then cut from the gel, taken through another step of PCR to amplify them, spliced into a cloning vector and then identified by sequencing and comparison to known sequences. Application of differential display methods have been used successfully for the identification and isolation of genes which are differentially expressed in cancers (Liang et al., 1992; Sager et al., 1993; Mok et al., 1994; Sun et al., 1994; Liang et al., 1994; Su et al., 1997; Shim et al., 1997; Burk et al., 1997; Zhang et al., 1997; Tikhonenko et al., 1996; Topol et al., 1997; Lewis et al., 1997; Zhang et al., 1993), heart disease (Utans et al., 1994; Russell et al., 1994), diabetes (Nishio et al., 1994), embryogenesis (Zimmermann and Schultz, 1994), developing brain (Watson and Margulies, 1993; Joseph et al., 1994), and growth factor stimulation (Hsu et al., 1993; Donohue et al., 1994). These techniques have been applied to the study of differential gene expression in the brain following development (Watson and Margulies, 1993), under a variety of disease states (Maratos-Flier et al., 1997; Noel et al., 1998) including brain tumors (Sehgal et al, 1998) and brain inflammation (Utans-Schneitz et al., 1999), long term potentiation (Matsuo et al., 1998), aging (Salehi et al., 1996), and response to drugs of abuse (Wang and Uhl, 1998; Lee et al., 1997; Ennulat and Cohen, 1997; Douglass et al., 1995; Douglass and Daoud, 1996; Couceyro et al., 1997).

RNA Isolation. For isolation of total RNA, MN9D cells can be extracted in TRIzol Reagent (BRL). RNA can be further purified by incubation with Dnase I (Promega) and phenol/chloroform, chloroform/isoamyl alcohol extractions. The amount of RNA can be quantitated by UV spectrophotometric methods and the quality of the RNA verified by running 1 μg of sample on a 1% denaturing agarose gel. For preparation of mRNA, the purified total RNA can be used as the starting material for use with the QuickPrep mRNA purification kit (Amersham Pharmacia). Amounts of purified mRNA can be determined by UV photometry. Quality of mRNA can be verified by running 1 μg of sample on a 1% denaturing agarose gel.

RT-PCR of Specific cDNAs. The poly A⁺ RNA isolated from MN9D cells can be used for first strand cDNA synthesis using an oligo(dT) primer and the SMART PCR cDNA Synthesis Kit (Clontech). Pairs of primers designed based on the mouse gene sequences for V-1, Nurr1, Ptx3 and TH can be synthesized. The PCR can be performed with Taq polymerase. The PCR conditions can be optimized for each set of primers (annealing temperature, MgCl₂ concentration, number of cycles, etc.). Controls, i.e. a commercial primer for control mRNA, a β-actin primer for mRNA from MN9D cell samples, and one negative control without reverse transcriptase, can be performed alongside all experimental samples. The cDNA fragments from RT-PCR reactions can be separated by electrophoresis on either agarose or acrylamide gels (depending on the size of the cDNA) for identification of the bands and then run on agarose gels for isolation of the cDNA. The corresponding bands can be cut out, extracted (QIAEX II Gel Extraction 150), and subcloned into an eukaryotic TA cloning vector, pCR3.1 (Invitrogen). Positive clones can be selected and the cDNAs purified (QIAGEN PlasmidMaxi Kit 25) and sequenced.

Northern Blot Analysis. Poly A+ RNA can be extracted from MN9D cells, resolved on a 0.8% denaturing agarose gel, and transferred to a Hybond-N nylon membrane by an electrophoretic method. The blot can be hybridized with ³²P-labeled probes. The blot can be exposed to X-ray film at either room temperature or −70° C. for desired periods of time. The probes specific for V-1, Nurr1, TH, and Ptx3 can be made from specific regions of the corresponding cDNA. Equal loading can be confirmed using a mouse GAPDH cDNA probe. Probes can be labeled with [α-³²P]dCTP (DECAprime II DNA labeling kit, Ambion).

Differential Display RT-PCR. Differential display experiments can be carried out using the DeltaDifferential Display Kit, Clontech. Briefly, poly A⁺ RNA from control or X61 protein-stimulated MN9D cells can be isolated as described above. Following reverse transcription, the cDNA can be amplified by PCR using pair-wise combinations of arbitrary “P” primers and “T” primers. The “T” primers are anchored oligo(dT) primers T₁₁VT, T₁₁VA, T₁₁VG and T₁₁VC (V=equimolar dA, dC, dG). The “P” primers represent common eukaryotic mRNA motifs. The dNTP mix used in the PCR can be supplemented with [α-³² P]DATP allowing the detection of low abundance products. The cDNAs can be analyzed on acrylamide and agarose gels for the detection of small and large PCR products, respectively (Liang and Pardee, 1992). X61 protein-stimulated PCR products can be excised from agarose gels, reamplified by PCR and subcloned into the PCR3.1 vector (Invitrogen) via the T/A cloning procedure. The inserts can then be sequenced. These constructs can be used for Northern blot analyses (for the confirmation of upregulation of the given mRNAs) as described earlier.

Suppression Subtractive Hybridization. Suppression subtractive hybridization can be carried out using the PCR-Select Subtraction Kit, Clonetech. Briefly, poly A⁺ RNA from MN9D cells can be isolated as described above. To identify X61 protein-stimulated genes, driver double stranded (ds) cDNA can be synthesized using mRNA template isolated from control or N18 protein-stimulated MN9D cells and tester ds cDNA can be synthesized using mRNA isolated from X61 protein-stimulated MN9D cells. To identify genes whose expression is decreased by X61 protein(s), the driver ds cDNA can be synthesized from mRNA from X61 protein-stimulated MN9D cells while tester ds cDNA can be synthesized from mRNA from both N18 protein- or distilled water-treated MN9D cells. Following first and second strand cDNA synthesis, the DNA samples can be digested with a blunt-ending restriction endonuclease (RsaI). The tester DNA can be divided into two portions, each of which is ligated to unique adaptors (adaptor 1, adaptor 2). These adaptors facilitate suppression PCR, which dramatically increases the probability of obtaining differentially expressed, rare transcripts. Excess driver ds cDNA can be hybridized with the tester ds DNA samples. After hybridization, the two samples can be mixed allowing the hybridization of tester single stranded (ss) DNA containing adaptor 1 with tester single stranded (ss) DNA containing adaptor 2. After hybridization, PCR reactions can be run with primers specific for regions in adaptor 1 and adaptor 2. The PCR products can be analyzed by polyacrylamide and agarose gel electrophoresis. X61 protein-stimulated PCR products can be excised from the gel, reamplified by PCR and subcloned into the PCR3.1 vector (Invitrogen) via the T/A cloning procedure. The inserts can then be sequenced at a sequencing facility. Amplified inserts can also be ³²P-labeled and used as probes for Northern blotting of mRNA obtained from the X61 protein-stimulated and control groups.

Western Blots. MN9D cells can be dissolved in 6 M Urea/0.25% SDS solution. In order to reduce viscosity, the samples can be freeze-thawed several times. Equal amounts of proteins can be separated by SDS polyacrylamide gel electrophoresis. The proteins can be transferred to nitrocellulose membrane and incubated in TBS/NP-40 (150 mM NaCl, 20 mM Tris pH 7.4, 5 mM KCl, 0.2% NP-40) containing 3% BSA. The blots can be incubated with antibodies (anti-Nurr1, anti-V-1, anti-Ptx3, and anti-TH) overnight. After extensive washes the blots can be incubated with HRPO-conjugated secondary antibodies for 30 minutes, followed by washes with TBS/NP-40. The immunoreactive bands can be detected with enhanced chemiluminescence.

### Example 3

### The Proteinaceous Composition is a Unique Factor

It is important to note that, in general, known trophic agents function as potent survival factors, i.e., they increase cell survival either in monolayer culture or in intact brain following injury to the nigrostriatal projection. However, the trophic factor of the present invention increases dopamine (DA) levels of individual DA neurons. Although, the factor may also act as a survival factor, it is clear that the factor of the invention regulates the levels of DA in a cell. It is contemplated that the factor may function by increasing the levels of DA cell at the level of biosynthesis and/or storage of the neurotransmitter and/or may regulate gene expression of proteins involved in DA synthesis and/or storage.

Analysis of Known Trophic Factors. The inventors contemplated that the factor may be similar in function to the 14-3-3 family of acidic, dimeric proteins which exert diverse influences on the signal transduction pathways of cells and participate in a variety of cell signaling processes that direct cell proliferation, differentiation and function (Fu et al., 2000). Since 14-3-3 is capable of activating both tyrosine and tryptophan hydroxylase (Itagaki et al., 1999: Ichimura et al., 1987) it is possible that the increase in DA observed in DA hybrid and primary cells might be secondary to the presence of the 143-3 family of proteins. Western blot analysis did, in fact, reveal the presence of 143-3 in the X61 cell. This is not surprising given the fact that 143-3 represents approximately 1% of soluble brain protein (Boston et al., 1982). In order to rule out the possibility that the effect of X61 protein(s) on MN9D dopamine is simply a function of the presence of one or more members of the 14-3-3 family, a direct experiment was conducted. Material obtained from X61 cells was exposed for 15 min at room temperature to an antibody against the 14-3-3 protein family and then was tested for activity. As can be seen in Table 3, X61 protein(s) exposed to antisera against 14-3-3 still retained its ability to-increase the DA content of MN9D cells by 2.3-fold in 24 hours.

TABLE 3
Lack of Effect of Anti-14-3-3 Antibody on the Dopamine Stimulatory
Effect of X61 Protein(s).

| Treatment | Control | X61 protein(s) | X61 protein(s) + anti |
|---|---|---|---|
| 14-3-3 | 12.73 ± 1.39 | 28.89 ± 0.96* | 28.37 ± 1.63* |

*p < 0.003 two tailed t test.

Experiment involved 24 hour incubation with X61 protein(s). X61 protein(s) was incubated with 100 microliter of concentrated monoclonal antibody against 14-3-3 (anti-14-3-3) obtained from Santa Cruz Biotechnology for 15 minutes at room temperature prior to addition to MN9D cells grown on 6-well plates. Data given as ng dopamine per well, n=3. The reason for the lower amounts of dopamine seen in this experiment as compared to that in Tables 2 and 3 is related to differences in time of exposure and plate size. X61 protein(s) were also preincubated with an antibody against neuronal nuclear protein as a control. This antibody had no effect on X61 protein(s) activity.

In addition to the 14-3-3 family, a host of other known neurotrophic factors were analyzed. Western blot analysis of X61 protein(s) did reveal the presence of low amounts of four of the known trophic factors: GDNF, ciliary neurotrophic factor (CNTF), transforming growth factor β2 (TGFβ2) and insulin growth factor II (IGF-II). However, none of these proteins had an effect on MN9D DA levels. MN9D cells exposed to GDNF (1 ng/ml for 5 days) actually had significantly lower (p<0.001) DA levels than controls (control, 467±18.4 ng/plate as compared to for GDNF treated cells, 356.0±14.4 ng/plate). Likewise, 24 hour treatment with 100 ng/ml of CNTF had no significant effect on MN9D DA levels (157.8±3.3 ng/plate for controls as compared to 156.5±10.9 ng/plate. Experiments conducted with TGFβ2 and IGF-II showed that these factors also were without effect on MN9D DA levels. No evidence for the presence in X61 supernatant of other trophic agents (Alexi et al., 2000) including brain derived neurotrophic factor (BDNF), neurotrophin 3, neurotrophin 4, insulin growth factor-I, fibroblast growth factor-1, bone morphogenetic factor-2, artemin, persephin, or neurturin was observed using Western blot analysis.

Thus, the X61 protein(s) differ not only in structure from known DA trophic agents, but also in respect to its mechanism of action on DA neurons. The known DA trophic agents exert their effects on DA neurons by increasing cell survival either in monolayer cultures or in the intact animal subjected to lesions of the DA nigrostriatal projection. The X61 protein(s) directly increases DA levels of individual MN9D cells (see Table 2) without increasing the cell numbers in the cultures. In the case of the three-fold increase in DA content of mesencephalic-tectal aggregates, where only some 20% of the dopaminergic neurons survive, it is conceivable that cell survival may be another effect of the factor. On the other hand, protein(s) from striatal-derived monoclonal cell lines increase by 2.4-fold the DA content of mesencephalic-striatal coaggregates which contain DA target cells and essentially all of the DA neurons survive (Heller et al., 1997).

### Example 4

### Activity of the Proteinaceous Composition in Primary Neurons

The use of the hybrid, dopaminergic MN9D cell line to detect an activity in X61 cells which increases dopamine is rapid and convenient. However, the MN9D hybrid cell is obviously not completely analogous to a dopaminergic neuron and for that reason an experiment was conducted to determine whether the active protein contained in the X61 supernatant had similar effects on primary mesencephalic dopaminergic neurons. For this purpose, the three-dimensional reaggregate culture system was utilized which provides sufficient dopaminergic neurons in culture for neurochemical analysis (Heller et al., 1993).

The three-dimensional reaggregate tissue culture system has proven to be useful for a wide variety of studies on monoaminergic and cholinergic neurons. The system was first described by Moscona (Moscona, 1961), and later applied to the CNS by Garber and Moscona (Garber and Moscona, 1972). This system has been utilized for studies on neuronal development, drug action and neurotoxicity (Heller et al., 1993; Atterwill et al., 1992; Honegger and Werffeli, 1988). The reaggregate culture system provides a method for the in vitro reconstruction of specific neuronal projections with circuitry similar to that observed in the intact brain. The neurons within such cultures exhibit developmental, pharmacological and toxicological responses closely mimicking those observed in vivo.

In this system it is possible to reconstruct the nigrostriatal projection by coaggregation of mesencephalic and striatal fetal cells. The nigrostriatal projection in culture can develop for up to 1 year to an adult morphological and neurochemical status with a time course essentially identical to that observed in the intact brain (Choi et al., 1993). For the purposes of these experiments however, the mesencephalon containing nigral dopaminergic neurons was coaggregated with tectum, a nontarget for the dopaminergic neuron. Under this Circumstance, the dopaminergic neurons do not form axons and approximately 80% of the neurons die off after 7 days in culture (Hoffmann et al., 1983) with the remaining cells appearing to form dense dendritic-like processes and probably surviving due to the formation of some autotopic connections. Such aggregates provide a model of dopaminergic degeneration for assessment of the effects of possible trophic factors.

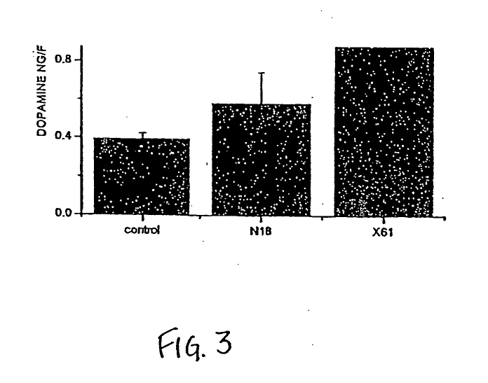

Mesencephalic-tectal aggregates were prepared from embryonic day 14 C57B1/6 mice using described methods (Heller et al., 1993). Approximately 5 million mesencephalic cells were co-cultured with 5 million tectal cells. Aggregates were treated with protein obtained from X61 cells or N18TG2 cells (0.3 mg/ml), or with distilled water (vehicle) every other day beginning on day 2 of culture for 20 days. As can be seen in FIG. 3, the dopamine content of aggregates exposed to protein from N18TG2 cells was not statistically different than vehicle controls. However, the dopamine content of mesencephalic-tectal aggregates exposed to X61 protein was nearly three-fold greater than dopamine levels in the control aggregates.

### Example 5

### Differentiation of Primary Neurons

The proteinaceous compositions or the dopaminergic stimulatory polypeptide factors of the invention that are obtained from immortalized clonal hybrid cells derived from embryonic murine corpus striatum are demonstrated to cause the differentiation of neurons. As described earlier, somatic cell fusion methods were used to immortalize neurons for the purpose of obtaining monoclonal cell lines expressing neurotrophic factors (Heller et al., 1992). Thus, monoclonal hybrid cells derived from neurons of the nigrostriatal projection expressing specific transmitter phenotypes were generated (Heller et al., 1992; Choi et al., 1991; Wainwright et al., 1995). The cells include a striatal cell line (X61) which is the source of the neurotrophic agents or proteinaceous agents of the invention (Heller et al., 2002) and a mesencephalic cell line (MN9D) expressing a dopaminergic (DA) phenotype which was used in various assays (Choi et al., 1991).

Cell lysates of the striatal X61 line comprise factors which have a stimulatory effect on both immortalized DA hybrid cells and on primary DA neurons (Wainwright et al., 1995). The DA stimulatory activity resides in a low molecular weight polypeptide fraction of less than 5 kDa.

The effect of this 5 kDa polypeptide fraction was examined on primary neurons in three-dimensional reaggregate culture which permits culture of mesencephalic DA and serotonergic (5-HT) in absence of appropriate target cells (Heller et al., 1992). In this situation, no axonal arbors are formed and the majority of monoaminergic neurons disappear presumably secondary to cell death. Some neurons survive and form fairly large processes which appear to be dendritic in character and make autotypic connections with other DA neurons. As described earlier, the X61 stimulatory factors increase DA content on DA neurons in such cultures (Heller et al., 2000). The present example describes the effect of the stimulatory factor on the morphology of both DA and 5-HT neurons by means of immunocytochemical methods.

Methods

The effects of the dopaminergic stimulatory polypeptide factors that were less than 5 kDa in size (UF4) were analyzed on dopamine (DA) and serotonin (5-HT) neurons in mesencephalic-tectal aggregates. A partially purified preparation (UF4) from X61 cell lysate, was added (20 μl/ml) to the aggregate culture medium from day 1 to day 15 of culture. Aggregate sections were examined for DA neurons using an antibody against tyrosine hydroxylase (TH) and for 5-HT neurons by antibody against 5-HT.

Results

In the case of the DA neurons, substantial numbers of densely stained DA neurons with extensive processes were observed. In contrast, while DA neurons were still present in the untreated controls, such dense groupings of heavily stained cells with extensive processes were at best extremely rare. UF4-treated aggregates also contain 5-HT neurons and axons which are more densely stained than the cells observed in the untreated controls. Neurochemical analysis of the aggregates and culture media revealed a 30% increase in aggregate DA (p<0.001) and a 52% increase in aggregate 5-HT following treatment with the UF4 partially purified preparation, Homovanillic acid a major metabolite of dopamine was increased by 75% (p<0.001) in the media from UF4-treated aggregates.

Differentiation of Neurons

Low molecular weight polypeptide fractions obtained from lysates of immortalized monoclonal cells derived from the striatum are therefore capable of increasing DA levels of both a monoclonal cell line (MN9D) (as shown in the previous Examples) and of primary DA and 5-HT neurons in three-dimensional reaggregate culture. In addition, the polypeptide fractions increase the immunocytochemical staining of both cell bodies and processes of these monoaminergic neurons. Purification and sequencing of the active polypeptides will permit assessment of their efficacy in the reversal of the motor dysfunction secondary to degeneration of the DA nigrostriatal projection. Thus, the low molecular weight polypeptide DA stimulatory factors due to their ability to regulate the DA phenotype are contemplated to be important for the treatment of Parkinsonism and other similar neurological disorders.

### Example 6

### Effect of the Proteinaceous Composition on Survival of Dopamine Neurons

As the number of dopaminergic neurons surviving in the cultures were not counted in the primary neuronal cultures it was not possible to determine if the effects was on cell survival or on dopamine levels in the remaining cells. The inventors are presently investigating this issue using cell counting methods (Vidal et al., 1995). Such aggregates also permit examination of effects of X61 protein on cells expressing cholinergic or γ-aminobutyric acid (GABA)ergic phenotypes.

Effects on Cell Survival. The inventors further contemplate experiments to analyze the effects of the factor on cell survival. In such experiments the DA cell number can be quantitated in aggregate cultures as well as in the intact animal subjected to nigrostriatal lesions following exposure to the X61 protein(s) combined with neurochemical analysis.

The increase in DA of coaggregates of primary DA neurons with nontarget or target cells can be examined in order to determine whether the increase in DA, in this case, is a function of a change in cellular DA content or an increase in either axonal proliferation or cell survival in the cultures.

Soluble protein obtained from X61 cells by washing the cells once with 10 ml of calcium and magnesium-free Tyrode's solution (CMF) and removing the cells from the plates with a cell scraper. The cells are collected from the plates with 20 ml of CMF, transferred to a 50 ml centrifuge tube, centrifuged at 1000×g for 3 minutes and the pellets stored at −80° C. The pellets are broken up in 1 ml of sterile distilled water containing a cocktail of agents to inhibit serine, cysteine, metalloproteases as well as calpain proteases (Protease-Arrest™) (GenoTechnology) with a 22 gauge needle on a syringe which is used to draw the pellet up into the syringe 10-15 times. This is followed by similar treatment with a 25 gauge needle. The material is then transferred to a 1 ml sterile microcentrifuge tube on ice and centrifuged at 9000×g for 30 minutes at 4° C. The supernatant is removed and transferred to a 15 ml centrifuge tube, which is vortexed and 100 μl removed for protein determination (Smith et al., 1985). The supernatant obtained is stored at −20° C.

For the assessment of stimulatory activity on dopaminergic MN9D cells, such cells are grown for 24 hours on 100 mm tissue culture plates and then X61 soluble protein or sterile distilled water (vehicle) added and the cultures incubated for either 24 or 48 hours. After this period, the MN9D cells on each plate are collected in 10 ml of CMF, transferred to 15 ml centrifuge tubes and centrifuged at 1000×g for 5 min. The supernatant is removed and 600 μl of 0.5N perchloric acid is added to the cell pellet which is then sonicated on ice for 10-15 seconds. The material obtained is centrifuged at 4000×g for 30 minutes and the supernatant stored at −80° C. MN9D cell DA content is determined by reverse phase high pressure liquid chromatography with electrochemical detection (HPLC-ED) as previously described (Heller et al., 1993). The pellet is stored at −20° C. for subsequent protein analysis.

Understanding the cellular mechanism(s) involved, in the case of the increase in DA produced by X61 protein(s) in three-dimensional aggregate cultures of primary neurons, requires quantitation of the number of DA neurons contained in the cultures. This can permit one to determine whether the effect is due to an increase in cellular DA content, or in DA cell survival. Such experiments are particularly pertinent in the case of the mesencephalic-tectal coaggregates which serve as a model of DA neuronal degeneration, since, in that case, the primary DA neurons are grown in the absence of target cells and the majority of neurons do not survive.

For the purposes of determination of the cellular nature of the X61 stimulatory effect, aggregates composed of mesencephalon with either tectum (a nontarget area) or striatum (a target area) can be prepared. The aggregates can be treated with soluble protein prepared either from X61 cells or N18TG2 cells (0.8 mg protein/ml), or with distilled water (vehicle) every other day beginning on day 2 of culture for 20 days. The effects of the protein(s) can be monitored by analysis of monoamine metabolites in the media and following 20 days of culture the aggregates can be collected for neurochemical and morphological analysis including cell counts and axonal arbor analysis as described below. In separate experiments, following 20 days of treatment with X61 protein(s), aggregates can be monitored for up to 6 months to determine the duration of the stimulatory effects on primary DA neurons.

Aggregate and Experimental Flask Preparation. The preparation of three-dimensional reaggregate cultures is based on the technique described by Garber and Moscona (Garber and Moscona, 1972). The general procedure and anatomical dissection of various brain regions is described in Heller et al., 1993). Utilization of such cultures for quantitative neurochemical and morphological analysis has been described by (Heller et al.-993; Heller et al., 1995).

Typically, one obtains sufficient tissue from 10 pregnant females at gestational day 14 to produce 18-20 flasks of mesencephalon co-cultured with another brain region resulting in flasks containing approximately 500 aggregates. The aggregates in flasks of similar culture combination are pooled and redistributed into appropriate numbers of experimental flasks. The pooling procedure simply insures that the samples are representative of the entire experimental group. The analytic unit for any given measure is a representative aliquot of pooled aggregates from each experimental group and each sample is a statistically independent replicate. The mixing of aggregates insures that each sample is representative of the whole flask and reduces variability (Won et al., 1992). Recent studies by the inventors using computer simulations of aggregate cultures have demonstrated the validity of the statistical approach and quantitative cell and morphological analysis.

Neurochemistry. Procedures for the processing of aggregate tissue and culture media as well as rat brain tissue for analysis of monoamine and metabolite content by high performance liquid chromatography (HPLC) are described in published methods (Heller et al., 1993; Schwartz et al., 1998).

Histochemistry. Procedures for the processing of aggregate cultures for immunocytochemical visualization of DA neurons and Falck-Hillarp-induced catecholamine histofluorescence (Heller et al., 1997).

Quantitation of Aggregate Morphometry and DA Cell Numbers. Computer-assisted image analysis methods for the quantitation of aggregates and neurochemically identified cells are described in detail in Heller et al. 1988; Vidal et al., 1995).

High Affinity DA Uptake. The extent to which DA neurons are able to accumulate exogenous transmitter can be utilized as an estimate of DA axonal processes within aggregates. The inventors have previously utilized this measure for ill vitro studies of the age-dependent development of mechanisms for the accumulation of DA and have demonstrated that 21-day-old aggregates accumulate 11 to 13 times as much DA as by 3 day cultures (Kotake et al., 1982). The developmental increase in accumulation of exogenous DA observed parallels the time course of increasing endogenous tissue levels of DA as well as the elaboration of DA axons and terminals. This index has also been used in studies of the neurotoxic effects of methamphetamine on DA neurons in aggregate culture which showed that drug-treated cultures accumulate exogenous DA to a much lesser extent than control, untreated cultures (Kontur et al., 1991). The reduction in exogenous transmitter accumulated was not due to a loss of DA cell bodies in the methamphetamine-treated cultures suggesting that the drug-inflicted damage was to DA axonal processes, a finding identical to that observed in vivo.

Quantitation of high affinity DA uptake per cell in MN9D cells and three-dimensional reaggregate culture can be conducted using a modification of the method described by Prochiantz et al., 1979. Such an analysis is possible since in the reaggregate culture system as well as in MN9D monolayer cultures the number of cells present can be determined. Cell numbers in monolayer cultures are estimated following trypsination by removal of the cells from the plate and cell counting in a hemocytometer. In aggregate culture quantitative estimates of the number of DA cells in a given sample is conducted by computer-assisted image analysis methods developed by the inventors (see above). The size of axonal arbor formed by a given number of DA cells can be quantitated using ³H-DA uptake or alternatively, by direct morphometric methods (Heller et al. 1997).

### Example 7

### Identification of the Proteinaceous Composition

One can isolate, purify and sequence the X61 protein(s) responsible for the increases in DA level observed in MN9D cells and primary DA neurons in coaggregate culture by the general methods set forth in the sections above and/or below with suitable modifications that one of skill in the art is well versed in.

It is clear that the X61 protein(s) are different from the known DA trophic factors. Therefore, isolation, purification and sequencing of the X61 protein(s) involved in the increases in DA level observed in MN9D cells and alterations of DA levels in coaggregates containing primary DA neurons are contemplated.

As the X61 protein(s) are obtained from X61 cells which are fusion products of a neuroblastoma they can be obtained in essentially unlimited quantities. For example, roller culture and glass carrier bead techniques can be used to facilitate large batch production of attached cultured cells. Analysis of increase in cellular in neurons can be used as a rapid assay method to follow activity during purification.

A typical preparation of crude X61 protein obtained from sixty 150 mm culture dishes of confluent X61 cells yields approximately 15 mg of starting material. Production of starting material can be scaled up from growing X61 cells on 150 mm culture dishes to using roller bottles which will result in a 4-fold increase to 60 mg of material. About 200 mg of starting material can easily be obtained from X61 cells and this represents the approximate amount of starting material utilized by those of skill in the art for the purification of neurotrophic factors (Lin et al., 1994; Smith et al., 1988).

Purification of Protein(s) from X61 Cells that Increase the DA Content of MN9D Cells

(A) Superdex 200. Aliquots (0.50 ml) of the soluble fraction obtained from osmotic lysis of X61 cells can be subjected to gel filtration chromatography on a Superdex 200 HR 10/30 column (1.0 cm×30 cm; Amersham Pharmacia Biotech) using the Amersham Pharmacia Fast Liquid Protein Chromatography (FPLC) system as described (He et al., 1998). Chromatography can be in phosphate-buffered saline (PBS) at 0.5 ml/min, and 0.5 ml fractions can be collected on ice. Corresponding fractions from multiple runs can be concentrated by ultrafiltration (Amicon) and assayed for the ability to increase the DA content of cultured MN9D cells. SDS-PAGE and silver staining of aliquots of fractions from a preliminary chromatographic separation has demonstrated that almost all detectable protein bands eluted discretely (within 2-3 fractions) and differently from each other. This indicates an effective purification step. Activity is expected to be maintained as the chromatography is relatively rapid (about 30 min) and in PBS, a medium in which the activity is stable. Peak activity fractions can be pooled, the NaCl concentration can be reduced to 0.05 M by dilution with phosphate buffer, and the sample can be frozen (−90° C.) after reconcentration using Amicon ultrafiltration.

(B) Mono 0 and/or MonoS. Ion exchange chromatography of the fraction from (A) above can be performed on a Mono Q and/or Mono S column, using the Amersham Pharmacia FPLC system (Gross and Kaplansky, 1983). One can then determine whether the activity can bind to each column at physiological pH and 0.05 M NaCl. If necessary, the pH can be lowered (to promote binding to Mono S) or raised (to promote binding to Mono Q). Elution of activity from each column can be done with a linear NaCl gradient, and corresponding fractions from multiple runs can be pooled. The NaCl concentration of each can be adjusted to 0.15 M. Fractions can be concentrated if necessary, and activity can be assayed as described in (A) above. Peak activity fractions can be pooled and frozen (−90° C.).

(C) Hydroxyapatite. If indicated, the activity from (B) can be chromatographed on a hydroxyapatite column and eluted with a linear sodium phosphate gradient as described (Gross and Kaplansky, 1983). Varied concentrations of sodium phosphate alone can also be tested, and, if necessary, eluted protein fractions can be diluted to reduce the phosphate concentration and the protein can be reconcentrated by ultrafiltration before assay.

(D) Reverse Phase Chromatrography and SDS-PAGE. Because it has been possible to utilize reverse phase chromatography and SDS-PAGE under non-reducing conditions as steps in the purification of other neurotrophic factors such as GDNF (Lin et al., 1994) and XTC-MIF (TGF-β3) (Smith et al., 1988), one can test whether the X61 activity (crude cytosol and/or partially purified as above) is stable to (a) 50% acetonitrile/50% (0.05% Trifluoroacetic acid in water) or (b) 0.1% SDS. Stability can be tested by assaying as above, following dialysis against several changes of PBS. If the activity is stable to condition (a), partially purified activity (above) can be injected onto a C8 reverse phase column and eluted with a linear gradient of 0 to 100% acetonitrile in 0.05% TFA in water, as described (Gross et al., 1996). Separated protein peak fractions (using the absorbance at 215 nm) can receive 1/50 volume of 10 mg/ml BSA (bovine serum albumin) as carrier and can be dialyzed against PBS before assay. Alternatively, protein peak fractions can be prepared for assay as described by Lin et al., 1994, using culture medium as carrier and serially diluting and re-concentrating with Centricon-10 concentrators (Amicon). If the activity is stable to condition (b), it can be subjected to SDS-PAGE under non-reducing conditions. One analytical lane can receive approximately 1-2 μg of sample and can be silver-stained (Gross et al., 1992), while the main sample can be run on one or more, parallel, preparative lanes. The latter can be sliced accordingly to the protein bands visible in the silver-stained gel, and the slices can be eluted by shaking at 4° C. overnight in 5 volumes of PBS containing 0.2 mg/ml BSA carrier. Eluates can be dialyzed, concentrated in Centricon-10s, and then assayed.

It is possible that the above purification scheme may yield a highly purified but non-homogeneous protein product, especially if the factor is a trace protein in the X61 cells. However, comparison of eluted fractions from each column chromatography by SDS-PAGE and silver staining with the activity peak can permit one to infer what protein band (or bands) co-purifies with the DA increasing activity. A silver stained band that co-purifies with the activity can be cut from a nitrocellulose electro-transfer and micro-sequenced and the amino-terminal sequence of the X61 factor can be obtained. A protein sequence data-bank search can then be used to determine whether or not this protein has been cloned and sequenced. If the protein sequence is unknown, the sequence can be used to synthesize oligo-deoxynucleotide(s) for cloning. If the protein is N-blocked, it can be trypsinized, subjected to reverse phase chromatography (C18 column), and major peptides can be micro-sequenced.

### Example 8

### Characterization of the Purified Factor(s) from X61 Cells

Presence of Multimers. A number of characterized neurotrophic factors are heterodimers that are inactivated by reduction and dissociation of the subunits. One can test whether or not the factor(s) from X61 cells is similarly sensitive by warming it (purified protein as well as cruder fractions) for 30 min at 50° C. in 50 mM dithiothreitol plus 50 mM Beta-mercaptoethanol (in a small volume and with 0.2 mg/ml BSA as carrier) followed by assay with comparison to a control sample that will be warmed but not reduced. Potential changes upon reduction of purified factor will be monitored by SDS-PAGE analysis and silver staining.

Characterization of Active Domain(s). Whether the X61-derived factor is dimeric or monomeric, it will be useful to test whether a portion of the intact protein will retain fall (or at least partial) activity. This can be tested by predetermining conditions of proteolysis (trypsin followed by saturating trypsin inhibitor or chymotrypsin followed by phenylmethylsulfonyl fluoride) that can lead to discretely smaller sizes, as measured by SDS-PAGE and silver staining as previously described (Gross et al., 1996). Aliquots of the factor sufficient for assay can then be similarly digested and tested for activity.

In addition, once the sequence of the protein(s) is obtained it can be compared to that of functionally similar proteins, for which there is structural information that may indicate potential active sites. Synthetic polypeptides, homologous to these sites on the protein, can be synthesized in a peptide and sequencing core laboratory and tested versus random-sequence polypeptide controls for the ability to block the bioactivity of the X6]-derived factor in tissue culture.

### Example 9

### Animal Model Studies of Therapeutic Effects

To examine the effects of X61 protein(s) on the morphological and functional status of DA neurons in the intact animal following experimentally induced neuronal injury. The therapeutic effects of X61 protein(s) on DA neurons in the intact animal or human subject will be facilitated by the availability of partially isolated and/or purified active protein(s) in sufficient quantities to permit repeated intracerebral or other forms of administration.

Animal Models of Disease

One can examine a variety of effects of the protein(s) of the invention on DA neurons in animal models of cell loss using both neurochemical analysis and morphological cell counting methods. In addition, the functional consequences of such effects will be examined by assessment of the ability of X61 protein(s) to reverse the effects of experimental damage to the DA system on extrapyramidal motor function.

Examination of the Effects of X61 protein(s) on the Morphological and Functional Status of DA Neurons in the Intact Animal Following Neuronal Injury. As the protein(s) factors of the invention have stimulatory effects on DA levels in primary DA neurons in three-dimensional reaggregate culture either in combination with target striatal cells or in the presence of nontarget tectal cells in which case there is a loss of a large number of DA neurons. This situation is analogues to a culture model of parkinsonian DA degeneration. As the X61 protein(s) are capable of producing a more than three-fold increase in DA in such cultures they are capable of reversing either experimental or disease induced functional losses secondary to DA degeneration in the intact animal. Before human clinical trials it will be necessary to examine the therapeutic ability of the X61 protein(s) to affect experimentally induced DA degeneration in animal models.

In order to assess the possible utility of the X61 striatal cell line derived protein(s) on DA function following experimental damage one can use intracerebral 6-hydroxydopamine (6-OHDA) to produce selective destruction of DA projections within the medial forebrain bundle as described by Chang et al. (1999). The adult male rats can be anesthetized with a mixture of ketamine (75 mg/kg), acepromazine (0.75 mg/kg) and rompun (4 mg/kg). Eight microgram (free base weight) of 6-OHDA in 2 μl of 0.2% ascorbic acid with 0.9% normal saline can be infused at a rate of 0.5 μl/min at the following coordinates: AP −4.4 mm, ML 1.2 mm relative to bregma, and DV −7.5 mm from the dura. After injection of 6-OHDA, the cannula can be left in place for 5 min before slowly retracting it. To prevent destruction of noradrenergic neurons, desipramine (12.5 mg/kg i.p.) is administered 30 min prior to the infusion of 6-OHDA. In the same surgical session, rats can be implanted with a 22 gauge guide cannula in the left striatum at coordinates relative to bregma and dura: AP, +1.0 mm; ML, 3.0 mm; and DV 4.0 mm. The cannula can be fixed to the skull with dental cement.

Two measures of DA function, forepaw placement and drug-induced rotation can be used to assess the effect of X61 protein(s) in either cell saving or functional recovery as has been done with a number of other trophic agents. Two weeks after 6-OHDA or sham lesion, rats can be examined for forepaw adjusting steps as described by Chang et al. (1999). For these experiments, the rats are moved across the table at a speed of 90 cm/12 seconds. Rats are held at the rear part of the torso by one hand with their hindlimbs lifted and one forepaw held steady with another hand of the experimenter so as to bear weight on the other forepaw. During this interval, the number of adjusting steps of the weight-bearing forepaw to compensate for the movement of the body is counted. The speed of the belt on the treadmill is controlled by a d.c. servomotor (Bison Gear and Engineering Corp., Downers Grove, Ill.) and a power controller unit (Motor Master, Glendale, Calif.). The belt of the treadmill is made of flexible rubber and the surface is covered with cloth tape to give a textured surface for the forepaw movements. Each stepping test consists of five trials for each forepaw, alternating between forepaws. In all experiments, the average of the five trials for each forepaw is used for analysis. Since forehand steps are more sensitive than other restorative measures, one can restrict the analysis to this variable.

Drug-induced rotations can be measured after forepaw testing using an automated rotometer consisting of a rotation bowl and a tether attached to the torso of the rat (San Diego Instruments, San Diego, Calif.). D-amphetamine-induced rotation (5 mg/kg, i.p.) can be determined three weeks after the 6-OHDA lesion in control and X61-treated animals following forepaw placement testing. The total number of rotations occuring during a one hour test period can be used for analysis. As demonstrated by Chang et al. (1999), the degree of striatal dopamine depletion is correlated with the average number of contralateral forehand adjusting steps after 6-OHDA lesion. A deficit in adjusting steps appears at a threshold of approximately 80% depletion. Similarly, significant drug-induced rotations are observed in response to amphetamine when dopamine is depleted by more than 80-85%.

Rats which exhibit a significant deficit in forepaw adjusting steps and/or significant, increase in amphetamine-induced rotations can be used for testing the ability of X61 protein(s) to reverse these behavioral measures. The animals selected following behavioral screening can receive multiple doses of protein(s) derived from either the X61 cell line or a control cell line (N18TG2) or vehicle 4 weeks after lesioning using a method similar to that described for GDNF by Rosenblad et al. (1998). A total of 10 repeated injections of either X61 protein or control protein (5 μg in 2 μl injection volume) or vehicle can be administered every second day for 20 days via a 28 gauge injection cannula connected with plastic tubing to a 10 μl Hamilton syringe. Injections can be made under halothane anesthesia by introducing the injection cannula through the guide cannula and infusing the protein(s) or vehicle at a rate of 0.5 μl/min, leaving the cannula in place for another 2 min before retracting.

Rats can be re-tested for forepaw placement and d-amphetamine-induced rotation at 1, 4 and 8 weeks after treatment with X61 protein(s). One day following behavioral testing, the rats can be killed and their brains used for neurochemical or morphological analyses. Following unilateral lesions striatal levels of tyrosine hydroxylase activity, DOPA decarboxylase activity and DA as previously described (Moore et al., 1971) can be examined in animals treated with control and X61 protein(s) (n=8 animals per group). One can also conduct quantitative experiments to determine the number of DA neurons in the X61 treated and control groups (n=8 animals/group). For the purposes of these experiments, rats can be anesthesized as described above for lesioning, then perfused with saline and 4% paraformaldehyde and processed for immunocytochemical visualization of dopaminergic neurons. Tyrosine hydroxylase positive cells in the various subdivisions of mesencephalon (substantia nigra versus ventral tegmental area) can be counted using stereological methods which avoid problems of double counting of cells in brain which can bias the results of sectional counting (West, 1999; Gundersen et al., 1988).

These experiments on the effects of X61 protein(s) on sparing of the degenerative and functional consequences of experimental damage to the nigrostriatal projection will provide essential information as to the potential of the X61 protein(s) as a candidate therapeutic agent in. Parkinson's disease, a model which has been extensively applied in the case of the other known DA trophic agents. Human clinical studies and treatment protocols will follow based on results of the animal studies such as those described above.

### Example 10

### Identification and Expression of Nucleic Acids Encoding the Proteinaceous Composition

Cloning

After obtaining amino acid sequence for the purified X61 protein, one can use a combination of PCR and library screening techniques to clone the fall length cDNA. Methods for cloning are well known to the skilled artisan and are also detailed in Sambrook, et al., (1989), incorporated herein by reference. In the first step, one can use PCR methods to amplify a specific cDNA sequence out of a population of single stranded cDNAs prepared by reverse transcription of mRNA isolated from X61 cells using any method known in the art or using any of the commercially available reagents and kits for this purpose (for example, the SuperScript Preamplification System, Life Technologies GIBCO BRL). One can synthesize a pool of degenerate oligonucleotide sense primers, based on at least 6 amino acids of sequence at the N-terminus, and a pool of degenerate oligonucleotide antisense primers, based on at least 6 amino acids of sequence at the C-terminus of the region of known amino acid sequence. The cDNA fragments from the PCR reaction can be separated by electrophoresis. Bands of the appropriate size can be cut out of the gels, extracted (for example, by QIAEX II Gel Extraction 150) and subcloned into a vector. One of skill in the art is familiar with vectors that may be used for this purpose Vectors include plasmids, cosmids, viruses (bacteriophage, animal viruses, and plant viruses), and artificial chromosomes (e.g. YACs). One of skill in the art would be well equipped to construct a vector through standard recombinant techniques (see, for example, Maniatis et al., 1988 and Ausubel et al., 1994, both incorporated herein by reference). In one specific example, one can use an eukaryotic vector such as the TA cloning vector, pCR3.1-Uni, that accepts products in the forward direction. The clones can then be screened with a guessmer probe derived from the sequence in the middle of the region of known amino acid sequence. Labeled clones can be sequenced to identify a clone with nucleotide sequence at the ends corresponding to the sequence of the primers, separated by a region of sequence which corresponds with the known amino acid sequence. The insert in this clone can then be labeled with ³²P and used to probe a brain-derived cDNA library (such as the Mouse Brain 5′-STRETCH PLUS cDNA Library from Clontech). If the cDNAs identified in the initial library screen are not to be complete sequences, one can re-probe the library with probes made from the ends of this identified cDNA to identify other partial overlapping cDNA products. Thus, one can obtain a complete open reading frame sequence by piecing together individual segments of the complete cDNA sequence. Alternatively, in place of the mouse brain cDNA library one could use a cDNA library made from mRNA isolated from the X61 cells. Such libraries can be obtained commercially. It is possible that the X61 protein may not be widely expressed throughout the brain and therefore would not be well represented in a cDNA library made from total brain mRNA. If the distribution of X61 protein is restricted to certain brain regions, then the probability for finding cDNA sequence for this protein would be higher in a cDNA library constructed with mRNA from X61 cells as the starting material.

a. Nucleic Acids

The present invention provides proteinaceous compositions that have trophic effects on dopaminergic neurons and increase dopamine content in cells. Described above are methods to clone nucleic acids encoding the proteinaceous compositions from the X61 cells. In some embodiments, the nucleic acid would comprise complementary DNA (cDNA). However, the nucleic acid may also be derived from genomic DNA, i.e., cloned directly from the genome of a particular organism. Also contemplated is a cDNA plus a natural intron or an intron derived from another gene; such engineered molecules are sometime referred to as “mini-genes.”

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given nucleic acid sequence encoding the X61 protein may be represented by natural variants that have slightly different nucleic acid sequences but, nonetheless, encode the same protein. Table 4 below provides a list of codons that can encode the same amino acids. The term “functionally equivalent codon” is used herein to refer to codons that encode the same amino acid, such as the six codons for arginine or serine (see Table 4, below), and also refers to codons that encode biologically equivalent amino acids, as discussed in the following pages.

In certain exemplary aspects, the present invention concerns nucleic acid sequences that encode proteins, polypeptides or peptides that are expressed in neuronal cells. It is contemplated that the nucleic acid segments of the invention, whether full length or partial gene sequences, are preferably isolated away from, or purified free from, total genomic DNA of cells or tissues. Included within the terms “nucleic acid and DNA segments”, are DNA and RNA segments and smaller fragments of such segments, and also recombinant vectors, including, for example, plasmids, cosmids, phage, viruses, and the like. A “DNA segment comprising an isolated or purified nucleic acid encoding the X61 derived protein” refers to a DNA segment including the coding sequences for the proteinaceous compositions described in the invention and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and smaller engineered gene segments that express, or may be adapted to express the proteins, polypeptides or peptides of the instant invention.

TABLE 4

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides encoding the X61 derived peptide, protein or polypeptide are contemplated.

The nucleic acids of the present invention include those encoding biologically functional equivalent proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

It will also be understood that nucleic acid sequences (and their encoded amino acid sequences) may include additional residues, such as additional 5 or 3 sequences (or N- or C-terminal amino acids), and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5 or 3 portions of the coding region or may include various internal sequences, i.e., introns, which are known to occur within genes.

Excepting intronic or flanking regions of any cancer marker gene, and allowing for the degeneracy of the genetic code, sequences that have between about 70% and about 80%; or more preferably, between about 81% and about 90%; or even more preferably, between about 91% and about 99% of nucleotides that are identical to the nucleotides of a disclosed sequence are thus sequences that are “essentially as set forth” in the given sequence.

An effective method for characterizing functional equivalence in nucleic acid sequences is hybridization. Nucleic acid sequences that are capable of hybridizing to one of the nucleic acid segments disclosed herein under relatively stringent conditions are functionally equivalent nucleic acid sequences. Suitable relatively stringent hybridization conditions will be well known to those of skill in the art and are further defined herein.

This invention thus particularly encompasses at least functional sequence analogs of the cloned sequences and nucleic acid sequences that are hybridizable to the cloned sequences.

Expression of the X61 Protein. After cloning the gene for the X61 protein, it can be subcloned into an expression vector. Expression vectors and systems are known in the art and are also described infra. One of skill in the art will recognize that any such expression vector can be used to express the protein of the invention. The protein can be then used for therapeutic purposes. Alternatively, one can use an expression vector to deliver the nucleic acid encoding the therapeutic protein of the invention for gene therapeutic purposes to a subject needing such a therapy.

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 5 lists non-limiting examples of elements/promoters that may be employed, in the context of the present invention, to regulate the expression of a RNA. Table 6 provides non-limiting examples of inducible elements, which are regions of a nucleic acid sequence that can be activated in response to a specific stimulus.

TABLE 5
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al.. 1989 |
| MHC Class II HLA-Dra | Sherman et al., 1989 |
| β-Actin | Kawamoto et al., 1988; Ng et al; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 6
Inducible Elements

| Element | Inducer | References |
|---|---|---|
| MT II | Phorbol Ester (TFA) | Palmiter et al., 1982; |
| | Heavy metals | Haslinger et al., 1985; Searle |
| | | et al., 1985; Stuart et al., |
| | | 1985; Imagawa et al., 1987, |
| | | Karin et al., 1987; Angel |
| | | et al., 1987b; McNeall et al., |
| | | 1989 |
| MMTV (mouse mammary | Glucocorticoids | Huang et al., 1981; Lee |
| tumor virus) | | et al., 1981; Majors et al., |
| | | 1983; Chandler et al., 1983; |
| | | Lee et al., 1984; Ponta et al., |
| | | 1985; Sakai et al., 1988 |
| β-Interferon | Poly(rI)x | Tavemier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | EIA, SV40 Large T Antigen | Taylor et al., 1989, 1990a, |
| | | 1990b |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et at, 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Repilcation

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Thus, therapeutic constructs of the present invention may be a viral vector that encodes for the proteins of the invention. Non-limiting examples of virus vectors that may be used to deliver a nucleic acid of the present invention include adenoviral vectors, adeno-associated viral vectors, retroviral vectors, lentiviral vectors etc.

Expression Systems. In one specific instance, the expression system may be a prokaryotic expression vector such as pTrcHis or pTrcHis2 (Invitrogen), which can be used to produce large quantifies of protein in E. Coli for purification. In such a system, the protein can be His-tagged at either the N- or C-terminal for case of purification of recombinant protein on a nickel-chelating resin. This system can also be used to make truncations of the X61 protein to examine which domains of the protein are necessary and sufficient to stimulate an increase in dopamine levels in MN9D cells. If the X61 protein is a complex folded protein (i.e., a homodimer with disulfide bonding) which is not properly processed in the E. Coli system, one can subclone the gene into pcDNA3.1/His and express the protein in a mammalian cell such as the HEK93 cell line. Both the overexpression and the His tag would make purification much simpler than purifying the protein from X61 cells. One of skill in the art will however recognize that one can use a variety of expression vectors and hosts to express and purify the protein. Non limiting examples of expression systems are described below.

Numerous expression systems exist that may be used to express the X61 derived protein compositions discussed above. Prokaryote- and/or eukaryote-based systems can be employed for use with the present invention to produce nucleic acid sequences, or their cognate polypeptides, proteins and peptides. Many such systems are commercially and widely available.

For example, the insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,879,236
U.S. Pat. No. 5,466,468
U.S. Pat. No. 5,871,986
U.S. Pat. No. 4,683,202
U.S. Pat. No. 5,925,565
U.S. Pat. No. 5,928,906
U.S. Pat. No. 5,935,819
Albeck et al., Neuroreport, 8(9-10): 2293-2298, 1997.
Alexi et al., Prog. Neurobiol., 60: 409-470, 2000.
Almendro et al., J. Immunol., 157(12): 5411-5421, 1996.
Andersen et al., Trends Pharmacol. Sci., 11: 231-236, 1990.
Angel et al., Mol. Cell. Biol., 7: 2256, 1987a.
Angel et al., Cell, 49: 729, 1987b.
Antibodies: A Laboratory Manual, Cold Spring Harbor Laboratory, Cold Spring Harbor Press, Cold Spring Harbor, N.Y., 1988.
Apfel, In: Clinical Applications of Neurotrophic Factors, 185-199, Lippincott-Raven, Philadelphia, 1997.
Atchison and Perry, Cell, 46: 253, 1986.
Atchison and Perry, Cell, 48: 121, 1987.
Atterwill et al., Neurotoxicology, 13(1): 39-53, 1992.
Ausubel et al., In: Current Protocols in Molecular Biology, Green Publishing Assoc., NY, 1987.
Balmforth et al., J. Neurochem., 47: 715-719, 1986.
Banerji et al., Cell, 27: 299, 1981.
Banerji et al., Cell, 35: 729, 1983.
Bannon and Roth, Pharmacol. Rev., 35: 53-68, 1983.
Beck, Prog. Neurobio., 44: 497-516, 1994.
Berkhout et al., Cell, 59: 273, 1989.
Blanar et al., EMBO J., 8: 1139, 1989.
Blusztajn et al., J. Neurosci., 12: 793-799, 1992.
Bodine and Ley, EMBO J., 6: 2997, 1987.
Boshart et al., Cell, 41: 521, 1985.
Boston et al., J. Neurochem., 38(5): 1475-82, 1982.
Bosze et al., EMBO J., 5: 1615, 1986.
Braddock et al., Cell, 58: 269, 1989.
Bulla and Siddiqui, J. Virol., 62: 1437, 1986.
Burk et al., EMBO J., 16(6): 1371-80, 1997.
Campbell and Villarreal, Mol. Cell. Biol., 8: 1993, 1988.
Campere and Tilghman, Genes and Dev., 3: 537, 1989.
Campo et al., Nature, 303: 77, 1983.
Capaldi et al., Biochem. Biophys. Res. Comm., 74(2): 425-433, 1977.
Carbonelli et al., FEMS Microbiol. Lett., 177(1): 75-82, 1999.
Carlsson, Neuropsychopharm., 1: 179-182, 1988.
Cashman, Int. Brain Res. Org. (Abstract 13), 1991.
Celander and Haseltine, J. Virology 61: 269, 1987.
Celander et al., J. Virology, 62: 1314, 1988.
Cepko, Annu. Rev. Neurosci., 12: 47-65, 1989.
Chandler et al., Proc. Natl. Acad. Sci. USA, 94(8): 3596601, 1997.
Chandler et al., Cell, 33: 489, 1983.
Chang et al., Neurosci., 88(2): 617-28, 1999.
Chang et al., Mol. Cell. Biol, 9: 2153, 1989.
Chatterjee et al., Proc. Nat'l Acad. Sci. USA., 86: 9114, 1989.
Childers, Neurochem. Res., 11: 161-171, 1985.
Choi et al., Brain Res., 552(1): 67-76, 1991.
Choi et al., Cell, 53: 519, 1988.
Choi et al., J. Neurosci. Methods, 46(3): 233-44, 1993.
Choi et al., Proc. Natl. Acad. Sci. USA, 89(19): 8943-7, 1992.
Choi-Lundberg et al., Science, 275(5301): 83841, 1997.
Cocea, Biotechniques, 23(5): 814-816, 1997.
Cohen et al., J. Cell. Physiol., 5: 75, 1987.
Costa et al., Mol. Cell. Biol., 8: 81, 1988.
Couceyro et al., Neuroreport, 8(11): 2437-41, 1997.
Crawford et al., J. Neurosci., 12: 3392-3398, 1992.
Cripe et al., EMBO J., 6: 3745, 1987.
Culotta and Hamer, Mol. Cell. Biol., 9: 1376, 1989.
Dandolo et al., J. Virology, 47: 55, 1983.
De Villiers et al., Nature, 312: 242, 1984.
Deschamps et al., Science, 230: 1174, 1985.
Donohue et al., J. Biol. Chem., 269(11): 8604-9, 1994.
Douglass and Daoud, Gene, 169(2): 241-5, 1996.
Douglass et al., J. Neurosci., 15(3Pt 2): 2471-81, 1995.
Edbrooke et al., Mol. Cell. Biol., 9: 1908, 1989.
Edlund et al., Science, 230: 912, 1985.
Ennulat and Cohen, Brain Res. Mol. Brain Res., 49(1-2): 299-302, 1997.
Eves et al., Proc. Natl. Acad. Sci. USA, 89: 4373, 4377, 1992.
Farfel et al., Brain Res., 578: 235-243, 1992.
Feng and Holland, Nature, 334: 6178, 1988.
Firak and Subramanian, Mol. Cell. Biol., 6: 3667, 1986.
Foecking and Hofstetter, Gene, 45: 101, 1986.
Fonnum, J. Neurochem., 24: 407-409, 1975.
Fu et al., Annu. Rev. Pharmacol. Toxicol., 40: 617-47, 2000.
Fujita et al., Cell, 49: 357, 1987.
Garber and Moscona, Dev. Biol., 27(2): 235-43, 1972.
Gerfen, Annu. Rev. Neurosci., 15: 285-320, 1992.
Gilles et al., Cell, 33: 717, 1983.
Gingrich and Caron, Annu. Rev. Neurosci., 16: 299-321, 1993.
Gloss et al., EMBO J., 6: 3735, 1987.
Godbout et al., Mol. Cell. Biol., 8: 1169, 1988.
Goodbourn and Maniatis, Proc. Nat'l Acad. Sci. USA, 85: 1447, 1988.
Goodbourn et al., Cell, 45: 601, 1986.
Grandy and Civelli, Curr. Opin. Neurobiol., 2: 275-281, 1992.
Graybiel and Ragsdale, In: Chemical neuroanatomy, Emson (Ed.), Raven, NY, 427-504, 1983.
Graybiel, Trends Neurosci., 13: 244-254, 1990.
Green et al., Proc. Natl. Acad. Sci. USA, 72: 4923-4927, 1975.
Greene and Tischler, Proc. Natl. Acad. Sci. USA, 73: 2424-2428, 1976.
Greene et al., Immunology Today, 10: 272, 1989.
Gross and Kaplansky, Biochim. Biophys. Acta, 740(3): 255-63, 1983.
Gross et al., J. Biol. Chem., 267(3): 2073-9, 1992.
Gross et al., J. Biol. Chem., 271(28): 16842-9, 1996.
Grosschedl and Baltimore, Cell, 41: 885, 1985.
Gruneberg, J. Hered., 34: 89-92, 1943.
Gundersen et al., APMIS, 96: 379-394, 1988.
Hammond et al., Brain Res., 512: 190-200, 1990.
Hammond et al., Science, 234(4781): 1237-40, 1986.
Haslinger and Karin, Proc. Nat'l Acad. Sci. USA., 82: 8572, 1985.
Hauber and Cullen, J. Virology, 62: 673, 1988.
He et al., J. Biol. Chem., 273(33): 20737-43, 1998.
Hefti, Annu. Rev. Pharmacol. Toxicol., 37: 239-267, 1997.
Heller et al., Clin. Exp. Pharmacol. Physiol., 22(5): 375-8, 1995.
Heller et al., In Progress in Parkinson's Disease Research, Hefti and Weiner (Eds.), 403-426, Futura Publishing Co. Inc., NY, 1992.
Heller et al., In: In Vitro Biological Systems: Preparation and Maintenance, Methods in Toxicology, Tyson and Frazier (Eds), Academic Press, New York, 27-45, 1993.
Heller et al., J. Comp. Neurol, 384(3): 349-58, 1997.
Heller et al., J. Neurosci. Methods, 25(1): 83-90, 1988.
Heller et al., Neurosci. Lett., 295: 1-4, 2000.
Hemmendinger et al., Brain Res., 222(2): 417-22, 1981.
Hemmendinger et al., Proc. Natl. Acad. Sci. USA, 78(2): 1264-8, 1981.
Hen et al., Nature, 321: 249, 1986.
Hensel et al., Lymphokine Res., 8: 347, 1989.
Herr and Clarke, Cell, 45: 461, 1986.
Hirochika et al., J. Virol., 61: 2599, 1987.
Hirsch et al., Mol. Cell. Biol, 10: 1959, 1990.
Hoffmann et al., Brain Res., 274: 275-281, 1983.
Holbrook et al., Virology, 157: 211, 1987.
Honegger and Werffeli, Experientia, 44(10): 817-23, 1988.
Horlick and Benfield, Mol. Cell Biol., 9: 2396, 1989.
Hornykiewicz and Kish, Adv. Neurol., 45: 19-34, 1987.
Hornykiewicz, Br. Med. Bull., 29: 172-178, 1973.
Hornykiewicz, In: The neurobiology of dopamine, Horn et al. (Eds.), Academic, London, 633-654, 1979.
Hsu et al., Biochem. Biophys. Res. Commun., 197(3): 1483-91, 1993.
Huang et al., Cell, 27: 245, 1981.
Hudson et al., Brain Res., 626(1-2): 167-74, 1993.
Hug et al., Mol. Cell Biol., 8(8): 3065-3079, 1988.
Hwang et al., Mol. Cell. Biol., 10: 585, 1990.
Ichimura et al., FEBS Lett., 219(1): 79-82, 1987.
Imagawa et al., Cell, 51: 251, 1987.
Imbra and Karin, Nature, 323: 555, 1986.
Imler et al., Mol. Cell. Biol., 7: 2558, 1987.
Inouye and Inouye, Nucleic Acids Res., 13: 3101-3109, 1985.
Itagaki et al., Biochemistry, 38(47): 15673-80, 1999.
Ivins et al., Mol. Pharmacol., 39: 531-539, 1991.
Jakobovits et al., Mol. Cell. Biol., 8: 2555, 1988.
Jameel and Siddiqui, Mol. Cell. Biol., 6: 710, 1986.
Jaynes et al., Mol. Cell. Biol., 8: 62, 1988.
Johnson et al., Mol. Cell. Biol., 9: 3393, 1989.
Joseph et al., Biochem. Biophys. Res. Commun., 201(3): 1227-34, 1994.
Kadesch and Berg, Mol. Cell. Biol., 6: 2593, 1986.
Karin et al., Mol. Cell. Biol., 7: 606, 1987.
Katinka et al., Cell, 20: 393, 1980.
Katinka et al., Nature, 290: 720, 1981.
Kawamoto et al., Mol. Cell. Biol., 8: 267, 1988.
Kebabian and Calne, Nature, 227: 93-96, 1979.
Kiledjian et al., Mol. Cell. Biol, 8: 145, 1988.
Klamut et al., Mol. Cell. Biol., 10: 193, 1990.
Koch et al., Mol. Cell. Biol., 9: 303, 1989.
Kontur et al., Brain Res., 428(1): 7-14, 1987.
Kontur et al., Neurosci. Lett., 129(2): 254-8, 1991.
Kordower et al., Proc. Natl. Acad. Sci. USA, 91(19): 9077-80, 1994.
Kotake et al., J. Neurosci., 2(9): 1307-15, 1982.
Kraus et al., FEBS Lett., 428(3): 165-170, 1998.
Kraus et al. FEBS Lett., 428(3): 165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3: 325, 1983.
Kriegler et al., Cell, 38: 483, 1984a.
Kriegler et al., Cell, 53: 45, 1988.
Kriegler et al., In: Cancer Cells 2/Oncogenes and Viral Genes, Van de Woude et al. (Eds), Cold Spring Harbor, Cold Spring Harbor Laboratory, 1984b.
Kriegler et al., In: Gene Expression, Hamer and Rosenberg (Eds), NY, Alan R. Liss, 1983.
Kuhl et al., Cell, 50: 1057, 1987.
Kunz et al., Nucl Acids Res., 17: 1121, 1989.
Lareyre et al., J. Biol. Chem., 274(12): 8282-90, 1999.
Lareyre et al., J. Biol Chem., 274(12): 8282-8290, 1999.
Larsen et al., Proc. Nat'l Acad. Sci. USA., 83: 8283, 1986.
Lasley et al., Life Sci., 35: 1921-1930, 1984.
Laspia et al., Cell, 59: 283, 1989.
Latimer et al., Mol. Cell. Biol., 10: 760, 1990.
Lee et al., Biochem. Biophys. Res. Commun., 240(2): 309-13, 1997.
Lee et al., Biochem. Biophys. Res. Commun., 240(2): 309-13, 1997.
Lee et al., Brain Res. Dev. Brain. Res., 52(1-2): 219-28, 1990.
Lee et al., DNA Cell Biol., 16(11): 1267-75, 1997.
Lee et al., Mol. Endocrinol., 2: 404411, 1988.
Lee et al., Nature, 294: 228, 1981.
Lendahl and McKay, Trends Neurosci., 13: 132-136, 1990.
Levenson et al., Hum Gene Ther, 9(8): 1233-6, 1998.
Levine et al., Brain Res., 131(2): 215-25, 1977.
Levine et al., Brain Res., 194(1): 263-S, 1980.
Levine et al., Exp. Neurol., 81(2): 34058, 1983.
Levinson et al., Nature, 295: 79, 1982.
Lewinsohn et al., Biochem. Pharmacol., 27(14): 1857-63, 1978.
Lewis et al., Mol. Cell. Biol., 17(9): 4967-78, 1997.
Liang and Pardee, Science, 257(5072): 967-71, 1992.
Liang et al., Cancer Res., 52(24): 6966-8, 1992.
Liang et al., Proc. Natl. Acad. Sci. USA, 91(26): 12515-9, 1994.
Lin et al., J. Neurochem., 63(2): 758-68, 1994.
Lin et al., Mol. Cell. Biol., 10: 850, 1990.
Lindner et al., Exp. Neurol., 132: 62-76, 1995.
Lovenberg et al., J. Neurochem., 57: 1563-1569, 1991.
Luria et al., EMBO J., 6: 3307, 1987.
Lusky and Botchan, Proc. Natl. Acad. Sci. USA, 83: 3609, 1986.
Lusky et al., Mol. Cell. Biol., 3: 1108, 1983.
Macejak and Sarnow, Nature, 353: 90-94, 1991.
Mack et al., Dev. Brain Res., 59: 249-251, 1991.
Majors and Varmus, Proc. Natl. Acad. Sci. USA, 80: 5866, 1983.
Maniatis et al., Molecular Cloning, A Laboratory Manual, Cold Spring Harbor Press, Cold Spring Harbor, N.Y., 1988.
Maratos-Flier et al., Methods Mol. Biol., 85: 297-304, 1997.
Matsuo et al., Neurosci. Lett., 244(3): 173-6, 1998.
McNeall et al., Gene, 76: 81, 1989.
Mendez et al., J. Neurosurg., 92(5): 863-9, 2000.
Meyer et al., Cell Transplant, 8(1): 25-36, 1999.
Miksicek et al., Cell, 46: 203, 1986.
Minna et al., Genetics, 79: 373-383, 1975.
Mok et al., Gynecol. Oncol., 52(2): 247-52, 1994.
Monsma et al., Brain Res., 492: 314-324, 1989.
Monsma et al., Brain Res., 492: 314-324, 1989.
Monsma et al., J. Neurochem., 54: 1200-1207, 1990b.
Monsma et al., Proc. Natl. Acad. Sci. USA, 87: 6723-6727, 1990a.
Moore and Bloom, Annu. Rev. Neurosci., 1: 129-69, 1978.
Moore et al., Brain Res., 30(1): 119-35, 1971.
Mordacq and Linzer, Genes and Dev., 3: 760, 1989.
Moreau et al., Nucl. Acids Res., 9: 6047, 1981.
Moscona, Experimental Cell Research, 22: 455-475, 1961.
Muesing et al., Cell, 48: 691, 1987.
Ng et al., Nuc. Acids Res., 17: 601, 1989.
Nishio et al., Faseb J., 8(1): 103-6, 1994.
Noel et al., Faseb J., 12(15): 1725-30, 1998.
Nomoto et al., Gene, 236(2): 259-271, 1999.
Nomoto et al., Gene, 236(2): 259-271, 1999.
O'Malley et al., Biochemistry, 29: 1367-1371, 1990.
O'Malley et al., New Biol., 4: 1-9, 1992.
Olsson et al., J. Neurosci., 15(5 Pt 2): 3863-75, 1995.
Ondek et al., EMBO J., 6: 1017, 1987.
Ornitz et al., Mol. Cell. Biol., 7: 3466, 1987.
Palmiter et al., Nature, 300: 611, 1982.
Pech et al., Mol. Cell. Biol., 9: 396, 1989.
Pelletier and Sonenberg, Nature, 334(6180): 320-325, 1988.
Perez-Stable and Constantini, Mol. Cell. Biol., 10: 1116, 1990.
Picard and Schaffner, Nature, 307: 83, 1984.
Pinkert et al., Genes and Dev., 1: 268, 1987.
Ponta et al., Proc. Natl. Acad. Sci. USA, 82: 1020, 1985.
Porcher and Heller, J. Neurochem., 19(8): 1917-30, 1972.
Porton et al., Mol. Cell. Biol., 10: 1076, 1990.
Potter et al., J. Neurochem. 41: 188-194, 1983.
Prasad and Sinha, In Vitro, 13: 125-132, 1976.
Prochiantz et al., Proc. Natl. Acad. Sci. USA, 76(10): 5387-91, 1979.
Puck et al., J. Exp. Med., 103: 273-284, 1956.
Queen and Baltimore, Cell, 35: 741, 1983.
Quinn et al., Mol. Cell. Biol., 9: 4713, 1989.
Redondo et al., Science, 247: 1225, 1990.
Reisman and Rotter, Mol. Cell. Biol., 9: 3571, 1989.
Remington's Pharmaceutical Sciences, 15^{th} ed, pages 1035-1038 and 1570-1580, Mack Publishing Company, Easton, Pa., 1980.
Resendez Jr. et al., Mol. Cell. Biol., 8: 4579, 1988.
Ripe et al., Mol. Cell. Biol., 9: 2224, 1989.
Rittling et al., Nucl. Acids Res., 17: 1619, 1989.
Ronnett et al., Science, 248: 603-605, 1990.
Rosen et al., Cell, 41: 813, 1988.
Rosenblad et al., Neuroscience, 82(1): 129-37, 1998.
Russell et al., J. Clin. Invest., 94(2): 722-30, 1994.
Sager et al., Faseb J., 7(10): 964-70, 1993.
Salehi et al., Experientia, 52(9): 888-91, 1996.
Sambrook et al., In: Molecular Cloning: A Laboratory Manual, Cold Spring Harbor Laboratory Press, Cold Spring Harbor, N.Y., 1989.
Satake et al., J. Virology, 62: 970, 1988.
Saucedo-Cardenas et al., Proc. Nail. Acad. Sci. USA, 95(7): 4013-8, 1998.
Schaffner et al., J. Mol. Biol., 201: 81, 1988.
Schallert et al., J. Neural. Transpl. Plast., 3: 332-333, 1992.
Schwarting and Huston, Prog. Neurobiol., 49(3): 215-66, 1996.
Schwartz et al., Neuroendocrinology, 68(6): 374-85, 1998.
Searle et al., Mol. Cell. Biol., 5: 1480, 1985.
Seeman et al., Nature, 365: 441-445, 1993.
Seeman, Synapse, 1: 133-138, 1987.
Sehgal et al., Int. J. Cancer, 76(4): 451-458, 1998.
Sharp and Marciniak, Cell, 59: 229, 1989.
Shaul and Ben-Levy, EMBO J., 6: 1913, 1987.
Sherman et al., Mol. Cell. Biol., 9: 50, 1989.
Shim et al., Proc. Natl. Acad. Sci. USA, 94(13): 6658-63, 1997.
Sibley and Monsma, Trends Pharmacol. Sci., 13: 61-69, 1992.
Sidhu and Fishman, Biochem. Biophys. Res. Commun., 166: 574-579, 1990.
Sidhu and Kebabian, Eur. J. Pharmacol., 113: 437-440, 1985.
Sleigh and Lockett, J. EMBO, 4: 3831, 1985.
Smidt et al., Proc. Natl. Acad. Sci. USA, 94(24): 13305-10, 1997.
Smith et al., Anal. Biochem., 150: 76-85, 1985.
Smith et al., Development, 103(3): 591-600, 1988.
Sokoloff et al., Nature, 347: 146-151, 1990.
Spalholz et al., Cell, 42: 183, 1985.
Spandau and Lee, J. Virology, 62: 427, 1988.
Spandidos and Wilkie, EMBO J., 2: 1193, 1983.
Steffey et al., Eur. J. Pharmacol., 207: 311-317, 1991.
Stephens and Hentschel, Biochem. J., 248: 1, 1987.
Sternberger, In: Immunocytochemistry, Wiley, NY, 104-169, 1979.
Stoof et al., Neurosci., 47: 755-770, 1992.
Stuart et al., Nature, 317: 828, 1985.
Su et al., Proc. Natl. Acad. Sci. USA, 94(17): 9125-30, 1997.
Sullivan and Peterlin, Mol. Cell. Biol, 7: 3315, 1987.
Sun et al., Cancer Res., 54(5): 1139-44, 1994.
Swartzendruber and Lehman, J. Cell. Physiology, 85: 179, 1975.
Takebe et al., Mol. Cell. Biol, 8: 466, 1988.
Tavernier et al., Nature, 301: 634, 1983.
Taylor and Kingston, Mol. Cell. Biol., 10: 165, 1990a.
Taylor and Kingston, Mol. Cell. Biol., 10: 176, 1990b.
Taylor et al., J. Biol. Chem., 264: 15160, 1989.
Thiesen et al., J. Virology, 62: 614, 1988.
Tiberi et al., Proc. Natl. Acad. Sci. USA, 88: 9491-9495, 1991.
Tikhonenko et al., J. Biol. Chem., 271(48): 30741-7, 1996.
Topol et al., Mol. Cell Biol., 17(8): 4801-10, 1997.
Treisman, Cell, 42: 889, 1985.
Trojanowski et al., J. Histochem. Cytochem., 31: 1217-1223, 1983.
Tronche et al., Mol. Biol. Med., 7: 173, 1990.
Tronche et al., Mol. Cell. Biol., 9: 4759, 1989.
Trudel and Constantini, Genes and Dev., 6: 954, 1987.
Tsumaki et al., J. Biol. Chem., 273(36): 22861-22864, 1998.
Tsumaki et al., J. Biol. Chem., 273(36): 22861-22864, 1998.
Ungerstedt, Acta Physiol Scand. Suppl, 367: 69-93, 1971.
Utans et al., Proc. Natl. Acad. Sci. USA, 91(14): 6463-7, 1994.
Utans-Schneitz et al., J. Neuroimmunol., 92(1-2): 179-190, 1998.
Vannice and Levinson, J. Virology, 62: 1305, 1988.
Vasseur et al., Proc. Natl. Acad. Sci. USA, 77: 1068, 1980.
Vidal et al., J. Neurosci. Methods, 56: 89-98, 1995.
Wainer et al., In: Gene Therapy in the Nervous System, Gage and Christen (Eds), Springer-Verlag, Berlin, Heidelberg, 86-105, 1992.
Wainwright et al., J. Neurosci., 15: 676-688, 1995.
Wang and Calame, Cell, 47: 241, 1986.
Wang and Uhl, Brain Res. Mol. Brain Res., 53(1-2): 344-347, 1998.
Watson and Margulies, Dev. Neurosci., 15(2): 77-86, 1993.
Weber et al., Cell, 36: 983, 1984.
Weinberger et al. Mol. Cell. Biol., 8: 988, 1984.
West, Trends Neurosci., 22(2): 51-61, 1999.
Winkler et al., J. Neurosci., 16(22): 7206-15, 1996.
Winoto and Baltimore, Cell, 59: 649, 1989.
Won et al., Brain Res., 575(1): 6-12, 1992.
Wu et al., Biochem. Biophys. Res. Commun., 233(1): 221-226, 1997.
Wu et al., Biochem. Biophys. Res. Commun., 233(1): 221-226, 1997.
Yamakuni et al., Adv. Pharmacol., 42: 30-32, 1998.
Yutzey et al. Mol. Cell. Biol., 9: 1397, 1989.
Zhang and Medina, Mol. Carcinog., 8(2): 123-6, 1993.
Zhang et al., Oncogene, 14(13): 1607-10, 1997.
Zhao-Emonet et al., Biochem. Biophys. Acta, 1442(2-3): 109-119, 1998.
Zhao-Emonet et al., Biochem. Biophys. Acta, 1442(2-3): 109-119, 1998.
Zhou et al., J. Neurochem., 59: 1875-1883, 1992.
Zimmermann and Schultz, Proc. Natl. Acad. Sci. USA, 91(12): 5456-60, 1994.
Zivin and Waud, Life Sci., 30: 1407-1425, 1982.

Zurcher and Da Prada, J. Neurochem., 38(1): 191-5, 1982.

```
# SEQUENCE LIS
#TING
<160> NUMBER OF SEQ ID NOS: 6
<210> SEQ ID NO 1
<211> LENGTH: 20
<212> TYPE: DNA
<213> ORGANISM: Artificial Sequence
<220> FEATURE:
<223> OTHER INFORMATION: Description of Artificial
#Sequence: Synthetic
            Primer
<400> SEQUENCE: 1
tcactgctca tcctgtccac
#
#
# 20
<210> SEQ ID NO 2
<211> LENGTH: 20
<212> TYPE: DNA
<213> ORGANISM: Artificial Sequence
<220> FEATURE:
<223> OTHER INFORMATION: Description of Artificial
#Sequence: Synthetic
            Primer
<400> SEQUENCE: 2
gagcacatga tgtcaaaggc
#
#
# 20
<210> SEQ ID NO 3
<211> LENGTH: 26
<212> TYPE: DNA
<213> ORGANISM: Artificial Sequence
<220> FEATURE:
<223> OTHER INFORMATION: Description of Artificial
#Sequence: Synthetic
            Primer
<400> SEQUENCE: 3
tgggctatgg catctctgag tcagct
#
# 26
<210> SEQ ID NO 4
<211> LENGTH: 18
<212> TYPE: DNA
<213> ORGANISM: Artificial Sequence
<220> FEATURE:
<223> OTHER INFORMATION: Description of Artificial
#Sequence: Synthetic
            Primer
<400> SEQUENCE: 4
actgggaccc gcgcaggt
#
#
# 18
<210> SEQ ID NO 5
<211> LENGTH: 21
<212> TYPE: DNA
<213> ORGANISM: Artificial Sequence
<220> FEATURE:
<223> OTHER INFORMATION: Description of Artificial
#Sequence: Synthetic
            Primer
<400> SEQUENCE: 5
ctgtgcctat acgcccccat c
#
#
#21
<210> SEQ ID NO 6
<211> LENGTH: 29
<212> TYPE: DNA
<213> ORGANISM: Artificial Sequence
<220> FEATURE:
<223> OTHER INFORMATION: Description of Artificial
#Sequence: Synthetic
            Primer
<400> SEQUENCE: 6
gggatgctga ggttccrctc caggctcac
#
# 29
```

1. A composition comprising at least one proteniaceous factor that is isolatable from X61 cells and has an ability to increase the dopamine content in neurons.

2. The composition of claim 1, wherein the proteinacious factor is further defined as heat-labile.

3. The composition of claim 1, wherein the proteinacious factor is further defined as trypsin-sensitive.

4. The composition of claim 1, wherein the proteinacious factor is further defined as having a molecular weight of less than 100 Kda.

5. The composition of claim 1, wherein the neurons are further defined as dopaminergic neurons.

6. The composition of claim 1, wherein the neurons are further defined as primary dopaminergic neurons.

7. The composition of claim 1, wherein the neurons are further defined as immortalized dopaminergic neurons.

8. A method for providing therapy and/or preventing a condition caused by a deficiency of dopamine and/or a loss or injury of dopaminergic neurons comprising administering to a patient a composition comprising at least one proteniaceous factor that is isolatable from X61 cells and has an ability to increase the dopamine content in neurons.

9. The method of claim 8, wherein the proteinacious factor is further defined as heat-labile.

10. The method of claim 8, wherein the proteinacious factor is further defined as trypsin-sensitive.

11. The method of claim 8, wherein the proteinacious factor is further defined as having a molecular weight of less than 100 Kda.

12. The method of claim 8, wherein the condition is Parkinson's disease or schizophrenia.

13. The method of claim 8, wherein the administration is by intravenous, intra-portal, intra-arterial, intracerebral, or direct local injection.

14. The method of claim 8, wherein the administration is oral.

15. The method of claim 8, wherein the administration intracerebral.

16. The method of claim 8, wherein the composition is partially purified.

17. The method of claim 8, wherein the composition is substantially purified.

18. The method of claim 8, wherein the composition is isolated from cells in culture.

19. The method of claim 8, wherein the composition is produced synthetically.

20. The method of claim 8, wherein the composition is produced by recombinant methods.

21. The method of claim 8, wherein said composition is administered in conjunction with other therapeutic agents.
